(19) United States
(12) Patent Application Publication
(10) Pub. No.: US 2008/0206734 A1
(43) Pub. Date: Aug. 28, 2008

(54) HIGH SURFACE CULTIVATION SYSTEM WITH FILLER

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CINVENTION AG, Wiesbaden (DE)

(21) Appl. No.: 12/039,330

(22) Filed: Feb. 28, 2008

Related U.S. Application Data
(60) Provisional application No. 60/892,138, filed on Feb. 28, 2007.

(51) Int. Cl. A01N 1/02; C12M 1/00; C12N 5/06

(52) U.S. Cl. 435/1.1; 435/289.1; 435/302.1; 435/298.1; 435/383

(57) ABSTRACT

An exemplary embodiment of a reversibly closable vessel suitable for the cultivation of cells and/or tissues can be provided. The exemplary vessel can comprise at least one reversibly closable aperture in the vessel wall, at least one wall provided in the interior of the vessel, with the wall being an integral part of the vessel. The interior can be divided into at least two compartments, whereas the wall comprises at least one of a hole, tube-like hole, opening, or a partially porous structure, which facilitates the flow of fluids from one compartment to another. At least one of the compartments can comprise at least one filler.

### CROSS-REFERENCE TO RELATED APPLICATION(S)

The present invention claims priority of U.S. provisional application Ser. No. 60/892,138 filed Feb. 28, 2007, the entire disclosure of which is incorporated herein by reference.

### FIELD OF THE PRESENT INVENTION

The present invention relates to new culture vessels, and in particular to culture vessels comprising arrangement(s) at least two compartment in fluid communication, wherein one compartment comprises a filler. Further, the present invention relates to culture systems of an interconnected array of culture vessels.

### BACKGROUND INFORMATION

Culture vessels, like roller bottles, are widely used for cultivation of cells, particularly of mammalian cells. The main applications thereof can be growing of cells, producing of cellular products or virus particles. Typical processes may be related to processing of high density cell cultures, co-cultures, cell infection and sample dialysis. Typically, culture vessels like roller bottles are containers of cylindrical shape that enable the rotation of the bottle around its longitudinal axis. The bottles are generally may be filled with a liquid medium for cultivating cells and by continuous or semi-continuous rotation the liquid is keeping the inner wall of the bottle wetted for cell growth and allows the convection of the medium. Principally, culture vessels, like roller bottles, are not completely filled with the liquid medium. There is generally a gas phase that usually comprises half or even more of the volume. Moreover, established roller bottles provide normally a small screw cap either with a membrane or without a membrane to enable gas exchange to the environment. Screw caps without a membrane are not commonly closed completely to facilitate the aforesaid gas exchange. Rotation of the bottle usually carried out by using appropriate apparatus with rotating rollers that keep the bottle rolling.

In commonly used culture systems, the pH of the liquid medium has to be maintained accurately close to physiologic levels. This is for example assured by utilizing a buffering system in the tissue culture fluid, in conjunction with an incubator in which carbon dioxide (CO₂) can be provided at a specific rate (usually to keep a concentration of 5 to 7 volume percent within the atmosphere of the incubator). Inflow of CO₂ into the roller bottle may be achieved by partially open the screw cap or via the embedded membrane that allows the gas exchange. The CO₂ reacts with water to form a weak acid and a carbonic acid, which in turn inter-reacts with the buffering system to maintain the pH near physiologic levels.

However, existing solutions have significant drawbacks in terms of efficiency. For example, such solutions can be related to the low performance of cell densities or, respectively, with the yield of cells or cell products or cell by-products. One reason can be that the surface volume ratio within a system is limited because a specific minimum volume of the gas phase has to be kept in order to allow the supply and equilibrium of oxygen and carbon dioxide. Another aspect is that the surface of the roller bottle is used as an active surface, particularly for cells that are growing adherently or semi-adherently. With a given surface area the space for attachment of adherent or semi-adherent cells is limited by the existing bottle design. In addition, the exchange of liquid medium is required to provide nutritional agents for vital cell cultivation. Compared to controlled bioreactors or perfusion systems, a conventional roller bottle may use a regular partial or complete exchange or supplementation of the liquid medium or nutritional compounds as well as supplemental factors.

A significant increase of cell densities, cell activity, proliferation, production of cell products or by-products can therefore depend on the available surface area, quantity of nutritional compounds, oxygen and CO₂ equilibrium and, not limited to, also of the biologic nature of the use type of cell or cell line. Specifically for each individual cell type or cell line, there are some conditions that suppress the vitality or limit the total number of vital cells within a given culture system. Another significant factor is that a living cell also produces by-products that affect the vitality or productivity or proliferation or biologic function of the cell itself or the cell culture. Among those may be for example lactic acid that affects the pH of the culture system and sometimes is shifted toward non-physiologic acidic values with adverse effects to the culture system. Another significant known issue is that the convection of nutritional compounds and gas within the liquid medium has also a significant impact on cell growth and vitality particularly because suitable convection can improve the microenvironment for cells.

Existing solutions may focus on single aspects of the aforesaid explained array of shortcomings. For example, European Application EP 1 400 584 A2 focuses on a roller bottle design that has an improved sealing that is not reducing the venting function of a membrane cap. U.S. Patent Publication No. 2004/0029264 describes a multi-chamber roller bottle of two cylindrical chambers that are interconnected whereby one chamber contains fresh liquid medium and the second the actual cell culture, hence increasing the overall volume and space of the culture vessel but reducing the actual available cell culture volume. U.S. Patent Publication No. 2004/0211747 describes a roller bottle with helical pleats for increasing the surface and facilitating the rinsing of the liquid medium during the rotation to assure wetting of the complete surface. However, the increase of surface particularly can be beneficial for adherently growing cells but without any significant benefit for suspension cell cultures.

Furthermore, conventional solutions are based on increasing surfaces but not in parallel assuring sufficient supply of medium, gas and other compounds. It has been found that increase of only surfaces results in limited increase of cell numbers.

### SUMMARY OF EXEMPLARY EMBODIMENTS OF PRESENT INVENTION

One exemplary object of the present invention is to provide a culture vessel that may be useful for cultivation of cells, tissues or tissue-like cell cultures, organs or organ-like cell cultures, multicellular organisms for different purposes.

Another exemplary object of the present invention is to provide a cultivation system for the aforesaid objective, whereby the cultivation system can be used for batch processing, extended batch processing, in-line or continuous or perfusion processes.

A further exemplary object of the present invention is to provide a cultivation process for cultivation of cells, tissues or tissue-like cell cultures, organs or organ-like cell cultures, multicellular organisms for different purposes.

Yet another further exemplary object of the present invention is to provide a culture vessel that comprises a significant increase of available surface for adherent or semi-adherent growth of cell cultures, controllable and improved convection of the liquid medium and the nutritional compounds, and/or significant improvement of gas exchange and equilibrium of oxygen and CO₂ within the exemplary cultivation system.

Still another exemplary object of the present invention is to provide active surfaces that allow improved convection of fluids, exchange of compounds, removal of cell-by products and/or stabilization of physiologic conditions to allow for cultivation of high cell concentrations in the exemplary cultivation system.

One exemplary embodiment of the present invention relates to a reversibly closable vessel suitable for the cultivation of cells and/or tissues, which can include at least one reversibly closable aperture in the vessel wall, at least one wall in the interior of the vessel, the wall being an integral part of the vessel, and dividing such interior into at least two compartments. The wall can comprise at least one of a hole, tube-like hole, opening, or a partially porous structure, allowing the flowing through of fluids from one compartment to another, and at least one of the at least two compartments comprises at least one filler.

For example, the filler can be a particulate material as further described herein. According to another exemplary embodiment of the present invention, a system can be provided which comprise at least two vessels as described above, whereas the vessels are interconnected via at least one aperture in their vessel wall, and a cultivation process using such vessel or system, in which at least one type of cells, tissue, tissue-like cell cultures, organs, organ-like cell cultures, or multicellular organisms are cultivated in the presence of at least one fluid or solid medium necessary for growing and/or cultivating the aforesaid culture.

### BRIEF DESCRIPTION OF THE DRAWINGS

Further objects, features and advantages of the present invention will become apparent from the following detailed description taken in conjunction with the accompanying Figures showing illustrative embodiments of the present invention, in which:

FIG. 1 are basic exemplary culture vessel designs for use with exemplary embodiments of the present invention;

FIG. 2 are exemplary schematic illustrations of an exemplary embodiment of a vessel with a reversibly removable cap design according to the present invention;

FIG. 3 in an exemplary illustration of a first exemplary blade orientation towards a longitudinal axis of the vessel;

FIG. 4 in an exemplary illustration of a second exemplary blade orientation towards the longitudinal axis of the vessel;

FIG. 5 is an illustration of an exemplary embodiment of a network-like system of blades according to the present invention;

FIG. 6 in an exemplary illustration of a third exemplary blade orientation towards the longitudinal axis of the vessel;

FIG. 7 is a schematic illustration of a first exemplary helical arrangements of the blades according to the present invention;

FIG. 8 is a schematic illustration of a second exemplary helical arrangements of the blades according to the present invention;

FIG. 9 is a schematic illustration of a third exemplary helical arrangements of the blades according to the present invention;

FIG. 10 is a schematic illustration of exemplary cross sections of the vessel or convection arrangement having wave-like or undulating blades according to an exemplary embodiment of the present invention;

FIG. 11 is a schematic illustration of a first exemplary embodiment of removably fixed blades in a vessel or a blade holder according to the present invention;

FIG. 12 is a schematic illustration of a second exemplary embodiment of the removably fixed blades in a vessel or a blade holder according to the present invention;

FIG. 13 is a schematic illustration of an exemplary embodiment of the arrangement having perforated blades according to the present invention;

FIG. 14 is a schematic illustration of an exemplary embodiment of the arrangement having a blade holder with holes or capillaries in the blades providing a fluid connection between different sectors and outside of the convection arrangement;

FIG. 15 is a schematic illustration of an exemplary embodiment of the arrangement having holes connecting different sectors or compartment of the convection arrangement or vessel;

FIG. 16 is a schematic illustration of a first exemplary embodiment of the convection arrangement in a vessel having different arrangements of blades fixed to a blade holder;

FIG. 17 is a schematic illustration of a second exemplary embodiment of the convection arrangement in a vessel having different arrangements of blades fixed to the blade holder;

FIG. 18 is a schematic illustration of a third exemplary embodiment of the convection arrangement in a vessel having different arrangements of blades fixed to the blade holder;

FIG. 19 is a schematic illustration of an exemplary embodiment of the blade holder for holding a plurality of blades.

FIG. 20 is a schematic illustration of a fourth exemplary embodiment of the convection arrangement in a vessel having different arrangements of blades fixed to the blade holder;

FIG. 21 is a schematic illustration of an exemplary embodiment of the convection arrangement inserted into a roller bottle;

FIG. 22 is a schematic illustration of an exemplary embodiment of a section from a layered structure of the exemplary convection arrangement;

FIG. 23A is a schematic illustration of a first exemplary embodiment of a cogwheel like designs of the exemplary convection arrangement;

FIG. 23B is a schematic illustration of a second exemplary embodiment of the cogwheel like designs of the exemplary convection arrangement;

FIG. 23C is a schematic illustration of a third exemplary embodiment of the cogwheel like designs of the exemplary convection arrangement;

FIG. 23D is a schematic illustration of an exemplary embodiment of roller bottles, rendering them rotatable in a staple;

FIG. 24A is an illustration of an exemplary vessel having at least two compartments or sectors, with two sectors defined by concentric arrangement of cylinders;

FIG. 24 B is an illustration of an exemplary vessel having four sectors created by dividing the outer annular space into two compartments;

FIG. 25 is an illustration of an exemplary vessel having an inner structure comprising a plurality of sectors with apertures at the separating wall.

FIG. 26 is an illustration of an exemplary embodiment of a system comprising a plurality of connected culture vessels according to the present invention;

FIG. 27 is an illustration of an exemplary vessel having one of the compartments filled with a particulate filler material or carrier; and

FIG. 28 is an illustration of an exemplary embodiment having three compartments.

Throughout the Figures, the same reference numerals and characters, unless otherwise stated, are used to denote like features, elements, components or portions of the illustrated embodiments. Moreover, while the subject invention will now be described in detail with reference to the Figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

To overcome the drawbacks of the conventional systems, e.g., in order to increase the available active surface for gas and liquid media exchange in a cell culture vessel, the provision of fillers in, e.g., one of the compartments of a culture vessel.

According to one exemplary embodiment according to the present invention, a highly efficient cell culture vessel can be provided for cultivation of cells and/or tissues comprising at least one reversibly closable aperture in the vessel wall, at least one wall in the interior of the vessel, the wall being an integral part of the vessel, and dividing the interior into at least two compartments. The wall can comprise at least one of a hole, tube-like hole, opening, or a partially porous structure, facilitating the flowing through of fluids from one compartment to another, and at least one of the at least two compartments comprises at least one filler.

The exemplary embodiment of the vessel can comprise at least one wall in the interior of the vessel dividing the interior in at least two compartments, and can include at least one (e.g., tube-like) hole, an opening or an at least partially porous structure allowing the flowing through of fluids from one compartment to the other. The compartments can comprise at least one filler, e.g., preferably a particulate material as described herein.

Exemplary Vessel

In one exemplary embodiment, the vessel can have, preferably, a shape of a cylindrical body, although any other geometric embodiments that are rotation-symmetric or can be rotated or agitated with an appropriate apparatus maybe also suitable. In one further exemplary embodiment, the culture vessel can have the form of a conventional roller bottle. For example. at least one inner wall may be provided, dividing the inner space of the vessel into at least two compartments or sectors, whereas the wall facilitates a fluid communication between the two sectors.

The length and/or diameter of the vessel can be scaled to any desired and suitable size depending on the particular use. It may be preferred that the culture vessel, e.g., a vessel in the shape of a cylindrical body, can have a length larger than about 10 mm, preferably more than 5 cm, still further preferable larger than about 20 cm and yet further preferable larger than about 50 cm. Thus, it can be preferred that the vessel is cylindrical and has a length in the range of about 1 to 5,000 cm, further preferable in the range of about 2 to 320 cm, still further preferable from about 20 to 180 cm, yet further preferable from about 40 to 240 cm and still yet further preferable from about 60 to 120 cm

Moreover the cylindrical vessel can have a diameter in the range of about 1 to 1,000 cm, preferably in the range of about 2 to 100 cm, more preferably in the range of about 10 to 80 cm, still further preferable from about 20 to 60 cm and still yet further preferable from about 35 to 55 cm.

Exemplary ratios of diameter to length can be 0.1:50, further preferable about 1:2 and still further preferable larger than about 1:3.

The exemplary embodiment of the culture vessel can comprise at least one aperture, preferably, an aperture being reversibly closable. The aperture may serve as an inlet or outlet for liquid or gaseous media, and may be equipped with suitable means for sealing against leakage, valves etc., as conventionally known. It can be preferred that the aperture may be located at the base of the vessel. Thus, in case the culture vessel is in the shape of a cylindrical body, at least one aperture can be located on one lateral end that allows in particular the filling of a liquid medium and/or cell suspension, e.g., using a pipette. The opposite lateral end of the cylindrical culture vessel can be without an aperture. In a further exemplary embodiment, opposite lateral end can also comprise at least one aperture. The apertures may preferably be centered to the longitudinal axis of the cylindrical culture vessel. Depending on the particular application, the aperture shape may vary. Thus, the shape of the aperture can be rectangular and/or can have any other regular or irregular form. It may be preferable that the shape of the aperture is substantially round.

For example, any arrangement known in the art to reversibly close and open the aperture can be used. A closing like a screw cap can be employed. In such case, the culture vessel comprises preferably, an appropriate thread, for example by comprising a threaded neck. In further exemplary embodiments, the apertures have a neck upon which the screw cap is located. In certain exemplary embodiments, the vessel can be narrowed toward the aperture or the respective neck comprising the aperture, and in other exemplary embodiments, both lateral ends of the vessel may be narrowed. In further exemplary embodiments, the aperture may not be embedded into the lateral ends, and preferably, at the central body of the culture vessel. In further exemplary embodiments, more than one aperture can be comprised at the vessel body, optionally any combination of at least one lateral aperture and at least one aperture at the body of the vessel.

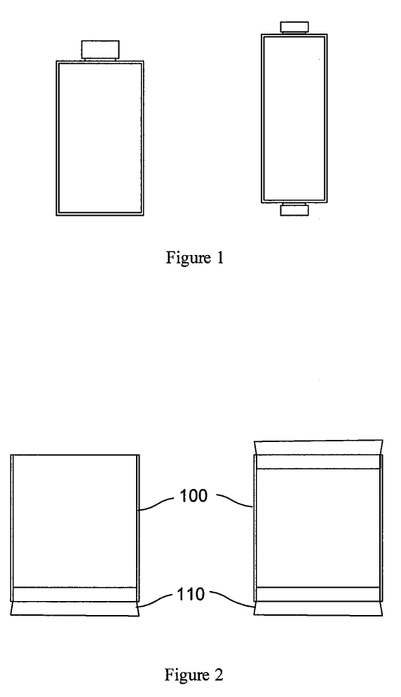

Exemplary culture vessel designs according to the present invention are shown in FIGS. 1 and 2.

In certain exemplary embodiments, at least one aperture and/or the closing of at least one aperture comprises a membrane for gas exchange as conventionally known, preferably with an appropriate sealing against leakage of the liquid medium. In other exemplary embodiments, the closing of at least one aperture can be opened to allow for gas exchange without using a membrane.

In further exemplary embodiments, the at least one aperture and/or the closing of the at least one aperture comprises a valve, either for unidirectional in-flow or out-flow of fluids such as liquids or gases or both, or bi-directional flow of fluids. Optionally more apertures and/or closings provide valves in any desired combination. The valves can be pressure-sensitive, or a modulating valve, and may be activated by mechanical arrangement, electromechanical arrangement, or magnetically, or by any appropriate arrangement or procedure conventionally known. In further exemplary embodiments, at least one closing comprises at least one aperture being either centric or eccentric. These apertures may also comprise closings that can be reversibly opened or closed, for example, screw caps, or valves, or the like, or any combination thereof. The closings used for any aperture can also comprise rotating joints or swivel couplings, optionally with valves, for example to connect a tube or tubing to the aforesaid apertures.

The vessel can be made from one part, or from multiple parts, optionally with modular parts that can be joined together. For example, in one embodiment, the body of the vessel is a cylindrical tube and the ends are caps that fit to the cylindrical tube and are connected without leakage of the liquid media. In certain exemplary embodiments, gaskets are used to assure appropriate sealing. In further exemplary embodiments, the parts are welded or bonded together by any conventionally known method. In further preferable certain exemplary embodiments, at least one of the caps can be joined and removed reversibly. FIG. 2 schematically illustrates an exemplary embodiment of a vessel 100 with a reversibly removable cap 110.

Exemplary Fillers

In a further exemplary embodiment, the cultivation vessel comprises at least one filler or a plurality of fillers. Such fillers comprise materials that increase the overall surface area of the cultivation system available for adherent cell growth, increase the surface area for equilibrium or exchange of fluids or fluid mixtures, and may include absorbents for absorbing fluids, fluid mixtures or a component or compound of a fluid or fluid mixture, or may include materials that provide a nutritional compound or a plurality of nutritional compounds or selectively adsorbs or desorbs physiologically or biologically active agents.

For example, the surface of the interior of the vessel is increased by the fillers by a factor of about 1.1 to 20·10¹⁰, more preferably of about 1.2 to 6·10¹⁰ and yet more preferably of about 2.0 to 5·10⁵.

Known fillers that increase the surface for adherent cell growth are micro- or macrocarriers, spherical particles, usually made out of cellulose, dextrane, gelatine, polystyrol, alginate, glass, carbon, ceramics or other organic, preferably polymeric materials, and the like, either chemically or biologically modified (or not). Suitable commercially available fillers can include, for example, Cytodex®, Cytopore®, Cultisphere®, Microhex®. Known drawbacks of such like fillers are that they can be designed to float in suspensions that are agitated in stirred tank systems or spinner systems, typically with actively controlled bioreactors. For conventional roller bottles, their usability is significantly limited, particularly because the agitation by simple rotation is insufficient to provide appropriate convection, aeration or gas exchange within the liquid phase, and moreover, rigid particle materials induce mechanical destruction of cells that are attached at the vessel wall. Another issue is that the presence of fillers like the previously named ones will only potentially increase the surface for adherent cell growth, a feature that is not useful for cells in suspension. Furthermore, previously described fillers may not comprise any function to align nutritional conditions. As described herein, sufficient growth prefers not only increase of effectively available surfaces but in parallel of increasing the nutritional conditions such like oxygenation, equilibrium of CO₂ and buffering and so forth.

According to the exemplary embodiments of the present invention, the suitable materials that increase the surface area for adherent cell growth, so called substrates or carriers, can be incorporated and beneficially utilized as fillers. In one certain exemplary embodiment, the discrete particles useful as substrates or carriers are provided within the inner vessel, whereby the vessel comprises only one compartment. In another exemplary embodiment, the substrates or carriers can be provided with one compartment of the vessel, preferably in a vessel with two compartments. Optionally, the carriers are provided in multiple compartments of the vessel with at least one compartment being free of a carrier material, as indicated in FIG. 27A. For example, the compartments of a blade holding system may be filled with those particles. One of the advantages of this exemplary embodiment is that the particles are filled to a substantially dense homogeneous packing without significant floating of the particles and without causing adverse shear stress, but optimally are exposed to the liquid medium and the gas phase or a beneficial fluid, fluid mixture or component or compound thereof, as shown in FIG. 27B. Moreover, this exemplary embodiment with densely packed particles for adherent cell growth can comprise a very high surface area for optimal contact between the carrier phase, the gas phase and the liquid medium phase.

The exemplary configuration of the vessel and the blades and respective blade holding system may be such like that at least two of the compartments are connected to each other and allow the exchange of at least the cultivation medium, preferably, also of the gas phases, or any other component or compound of the used fluid or fluid mixture or any combination thereof. At least one separating wall or one part of the blade being part of the compartment or sector with the packed carrier particles comprises the rinsing function. Embodiments with higher performance comprise a plurality of compartments filled with carriers, either inner compartments or outer compartments of the vessel, and continuously rinse the liquid and/or provide the exchange of a fluid, fluid mixture or component or compound of a fluid. Usually, in conventional use the carrier volume used conventionally is due to the aforesaid shortcomings limited to approximately 5-8% of the liquid culture volume. The inventive embodiment, allows increasing the carrier volume up to 90%.

In certain exemplary embodiments, the substrate or carrier mold is also a blade, blade holder or blade holding system or a plurality of the foregoing.

The exemplary structure of the filler/carrier can be porous, with ultramicro-porous, micro-porous or meso-porous or macro-porous or combined pores or porosities. The porous carrier or filler particles can comprise a gradient of different porous layers or sections in any desired geometric or three-dimensional direction. In certain exemplary embodiments, the porous structure is partially or completely a mesh-like porous structure or a lattice, and/or comprises a mesh-like trabecular, regular or irregular or random or pseudo-random, structure or any combination thereof or the aforesaid porous structures.

Exemplary Functionalized Fillers

The exemplary embodiment of the cultivation vessel can comprises at least one filler, e.g. a particulate filler, in at least one of the compartments/sectors of the vessel. Other fillers can be, for example, ion exchangers, those for binding positively charged ions or cations, which display on their surface negatively charged groups; and those for binding negatively charged ions or anions, which display on their surface positively charged groups. The ion exchanger can be composed of the solid support material, a liquid or gel, or any combination thereof, like for example a hydrogel or polymer composed for easily hydrated groups like cellulose consisting of polymers of sugar molecules. These materials consist of polymeric matrixes to which are attached functional groups. The chemistry of the matrix structure is polystyrenic, polyacrylic or phenol-formaldehyde, but not limited to. The functional groups are numerous, for example, but not limited to: sulfonic, carboxylic acids, quaternary, tertiary and secondary ammonium, chelating (thiol, iminodiacetic, aminophosphonic and the like). The various types of matrices and their degree of crosslinking translate into different selectivity for given species and into different mechanical and osmotic stability. Many resins and adsorbents can be obtained with a narrow particle size distribution for optimum hydrodynamic and kinetics properties. Ion exchange resins are also characterized by their operating capacities function of the process conditions. Ion exchange resins are mostly available in a moist beads form (granular or powdered forms are also sometime used, dry form is also available for applications in a solvent media) with a particle size distribution typically ranging about 0.3-1.2 mm (16-50 mesh) with a gel or macroporous structure. Ion exchangers can preferably be used as single or combined moulds made out of one single or multiple parts. In further exemplary embodiment, the ion exchanger comprises at least one blade or a blade holder or a blade holding system a plurality of blades or blade holders or blade holding systems. In further exemplary embodiments, the ion exchanger comprises a micro- or macro-carrier, structured carrier or carrier mold. In still further exemplary embodiments, the ion exchanger comprises both, i.e. a combination of at least one blade or blade holder or a blade holding system combined with a carrier or carrier mold.

Further useful fillers are absorbents to absorb at least one compound of the culture, of at least one fluid, fluid mixture or component of a fluid mixture or a combination thereof. Suitable absorbers, for example, are used to absorb proteins. For protein absorption Diethylaminoethyl (DEAE) or Carboxymethyl (CM) absorbers are appropriate. Since proteins are charged molecules, proteins in the cultivation system will interact with the absorber depending on the distribution of charged molecules on the surface of the protein, displacing mobile counter ions that are bound to the resin. The way that a protein interacts with the absorber material depends on its overall charge and on the distribution of that charge over the protein surface. The net charge on a given protein will depend on the composition of amino acids in the protein and on the pH of the fluid. The charge distribution will depend on how the charges are distributed on the folded protein. A person skilled in the art can determine the appropriate absorber or combination of absorbers and/or the pH of the fluid depending on the protein's isoelectric point for adjusting the absorption properties and function.

Other useful absorbers are gas absorbing materials, preferably for absorption of CO₂, oxygen, N₂, NO, NO₂, N₂O, and SO₂. beside absorbents known in the art, further useful absorbents could be selected from materials that comprise imidazolium, quaternary ammonium, pyrrolidinium, pyridinium, or tetra alkylphosphonium as the base for the cation, whereby possible anions include hexafluorophosphate [PF₆]—, tetrafluoroborate [BF₄]—, bis(trifluoromethylsulfonyl) imide [(CF₃SO₂)₂N]—, triflate [CF₃SO₃]—, acetate [CH₃CO₂]—, trifluoroacetate [CF₃CO₂]—, nitrate [NO₃]—, chloride [Cl]—, bromide [Br]—, or iodide [I]—, among many others. Any combination of a absorbing material can be selected with regard to the solubility of the relevant gas. Exemplary absorbers can facilitate a chemical interaction between the selected gas or gas mixture to be absorbed or a physical interaction, like the solution in an appropriate solvent. Suitable absorbers are also activated carbon or activated carbon-like materials, chelating agents such as penicillamine, methylene tetramine dihydrochloride, EDTA, DMSA or deferoxamine mesylate and the like.

The exemplary absorber can be provided as a liquid solution, gel, solid or any combination thereof. The solid can be composed of particles or a structured mold or any combination thereof.

In further exemplary embodiments, the absorber is embedded at least in one compartment of the vessel or a blade or a blade holder or a blade holding system or any combination thereof.

In further exemplary embodiments, the absorber also comprises a carrier or carrier mold, or an ion exchanger or any combination thereof.

Further beneficial fillers used in the exemplary embodiment of the present invention can comprise and/or have incorporated and/or are capable to release beneficial agents. Beneficial agents can be selected from biologically active agents, pharmacological active agents, therapeutically active agents, diagnostic agents or absorptive agents or any mixture thereof. Beneficial agents can be incorporated partially or completely into at least one compartment or a plurality of compartments or cavity or plurality of cavities of the vessel, a blade, a blade holder, a blade holding system, carrier mould, ion exchanger, absorber or any combination thereof. Biologically, therapeutically or pharmaceutically active agents according to the exemplary embodiment of the present invention may be a drug, pro-drug or even a targeting group or a drug comprising a targeting group. The active agents may be in crystalline, polymorphous or amorphous form or any combination thereof in order to be used in the present invention. Suitable therapeutically active agents may be selected from the group of enzyme inhibitors, hormones, cytokines, growth factors, receptor ligands, antibodies, antigens, ion binding agents like crown ethers and chelating compounds, substantial complementary nucleic acids, nucleic acid binding proteins including transcriptions factors, toxines and the like. Examples of therapeutically active agents are described in International Patent Publication WO 2006/069677 (see pages 36-44 thereof).

Suitable exemplary signal generating agents are materials which in physical, chemical and/or biological measurement and verification methods lead to detectable signals, for example in image-producing methods. It is not important for the exemplary embodiment of the present invention whether the signal processing is carried out exclusively for diagnostic or therapeutic purposes. Typical exemplary imaging methods are for example radiographic methods, which are based on ionizing radiation, for example conventional X-ray methods and X-ray based split image methods such as computer tomography, neutron transmission tomography, radiofrequency magnetization such as magnetic resonance tomography, further by radionuclide-based methods such as scintigraphy, Single Photon Emission Computed Tomography (SPECT), Positron Emission Computed Tomography (PET), ultrasound-based methods or fluoroscopic methods or luminescence or fluorescence based methods such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Signal generating agents and targeting groups can be selected from those as described in International Patent Publication WO 2006/069677 (see pages 12-36 thereof).

According to the exemplary embodiment of the present invention, and incorporation of the exemplary beneficial agents may be comprised by incorporating the aforesaid beneficial agents into at least one cavity or compartment or a plurality of cavities or compartments of the inventive vessel, blade, blade holder, blade holding system, filler, carrier, carrier mould, ion exchanger, absorber or any combination thereof. Incorporation may be carried out by any suitable mean, preferably by dip-coating, spray coating or the like or infusion of the beneficial agents directly into the aforesaid structures. The beneficial agent may be provided in an appropriate solvent, optionally using additives. The loading of these agents may be carried out under atmospheric, sub-atmospheric pressure or under vacuum. Alternatively, loading may be carried out under high pressure. Incorporation of the beneficial agent may be carried out by applying electrical charge to the implant or exposing at least a portion of the implant to a gaseous material including the gaseous or vapor phase of the solvent in which an agent is dissolved or other gases that have a high degree of solubility in the loading solvent. In further exemplary embodiments, the beneficial agents are provided using carriers that are incorporated into the compartment of the implant. Carriers can be selected from any suitable group of polymers or solvents.

Exemplary carriers may be polymers like biocompatible polymers, for example. In certain exemplary embodiments, it can be particularly preferred to select carriers from pH-sensitive polymers, like, for example, however not exclusively: poly(acrylic acid) and derivatives, for example: homopolymers like poly(amino carboxylic acid), poly(acrylic acid), poly(methyl acrylic acid) and their copolymers. This applies likewise for polysaccharides like celluloseacetatephthalate, hydroxylpropylmethylcellulose-phthalate, hydroxypropylmethylcellulosesuccinate, celluloseacetatetrimellitate and chitosan. In certain embodiments, it can be especially preferred to select carriers from temperature sensitive polymers, like for example, however not exclusively: poly(N-isopropylacrylamide-co-sodium-acrylate-co-n-N-alkylacrylamide), poly(N-methyl-N-n-propylacrylamide), poly(N-methyl-N-isopropylacrylamide), poly(N—N-propylmethacrylamide), poly(N-isopropylacrylamide), poly(N,N-diethylacrylamide), poly(N-isopropylmethacrylamide), poly(N-cyclopropylacrylamide), poly(N-ethylacrylamide), poly(N-ethylmethylacrylamide), poly(N-methyl-N-ethylacrylamide), poly(N-cyclopropylacrylamide). Other polymers suitable to be used as a carrier with thermogel characteristics are hydroxypropylcellulose, methylcellulose, hydroxypropylmethylcellulose, ethylhydroxyethylcellulose and pluronics like F-127, L-122, L-92, L-81, L-61. Preferred carrier polymers include also, however not exclusively, functionalized styrene, like amino styrene, functionalized dextrane and polyamino acids. Furthermore polyamino acids, (poly-D-amino acids as well as poly-L-amino acids), for example polylysine, and polymers which contain lysine or other suitable amino acids. Other useful polyamino acids are polyglutamic acids, polyaspartic acid, copolymers of lysine and glutamine or aspartic acid, copolymers of lysine with alanine, tyrosine, phenylalanine, serine, tryptophan and/or proline.

In certain exemplary embodiments, the beneficial agents comprise metal based nano-particles that are selected from ferromagnetic or superparamagnetic metals or metal-alloys, either further modified by coating with silanes or any other suitable polymer or not modified, for interstitial hyperthermia or thermoablation.

In certain exemplary embodiments, the beneficial agents comprise partially or completely the vessel, a single or plurality of blades, blade holders or blade holding systems, a carrier, a carrier mold, an ion exchanger or an absorber or any combination thereof.

In some most further exemplary embodiments, at least one beneficial agent comprises the structural body of the filler.

Exemplary Convection Arrangement/Blades

Additionally, the culture vessel comprises a convection arrangement inside the vessel that enables a convection and/or rinsing of a fluid within the vessel. The convection arrangement can include a magnetic stirring bar, agitator, stirrer, and/or at least one blade optionally fixed to a blade holder. In one exemplary embodiment, the convection arrangement can comprises at least one blade, which may be connected directly to the vessel, optionally connected to a blade holder to be inserted into a vessel, or a combination thereof. Such exemplary arrangement can be capable to generate convection in a fluid in case the fluid and/or vessel and/or the blade(s) are agitated. A blade may be designed to take up the liquid medium similar to a bucket wheel, particularly if the volume of the vessel is not completely filled with liquid medium, and/or to induce convection within the liquid phase during the agitation of the vessel and/or fluid. Preferably, the convection arrangement located within the vessel can comprise one blade, still more preferably two blades, or more than two blades.

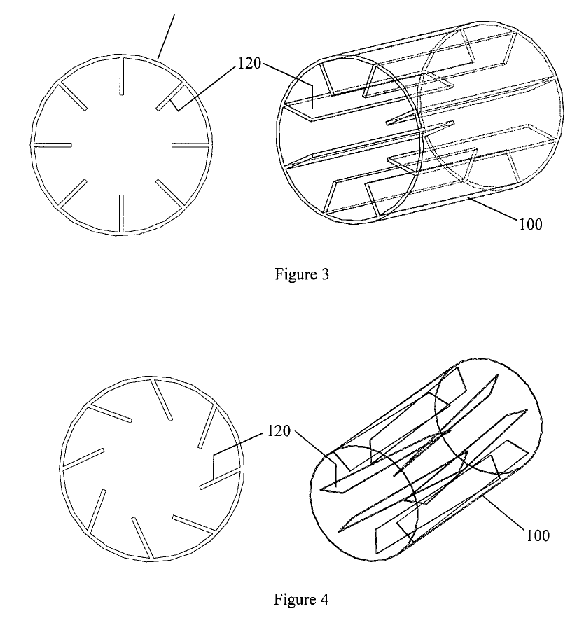

Blades 120 can have a parallel orientation towards the longitudinal axis of the vessel 100, e.g., 90° rectangular to the cross-sectional plane; examples for suitable blade orientations are shown in FIGS. 3, 4 and 6.

The blades can also have any different angles towards the rectangular or the longitudinal plane or both, preferably, about 0.1° to 179°, further preferable about 2° to 140°, still more preferable about 40° to 110°.

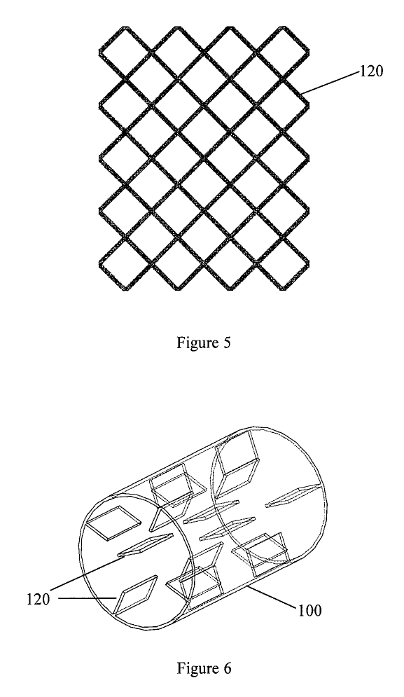

Furthermore, the blades can be completely connected to the inner vessel wall or only partially. In certain exemplary embodiments, at least one blade is fixed to a blade holder as defined below, in other exemplary embodiments, at least one blade can be only partially fixed to a blade holder as defined below, or movable. In any dimensional plane, a plurality of blades 120 can intersect at least one blade or another plurality of blades to provide a network structure, as illustrated for instance in FIG. 5.

The angle of intersections can be varied, and according to a plurality of blades intersecting another any individual variation can be realized. In certain exemplary embodiments, one plurality of non-intersecting, parallel blades that are parallel to the cross-sectional plane, intersect at least one blade or a plurality of blades that are not parallel to the cross-sectional plane. Furthermore, a single blade or a plurality of blades, either intersecting or not, can be designed to have individually different angles either in the rectangular or longitudinal plane or in any other plane or any combination thereof.

A single blade can have the length of the complete vessel body or a shorter length; in further exemplary embodiments, at least one blade is partially or completely discontinuous. Furthermore, the position of a single blade or a plurality of blades 120 can be at any suitable point or section or place within the inner vessel wall, as e.g., shown in FIG. 6. Thus, in certain exemplary embodiments, a plurality of blades is completely or partially discontinuous. The design of blades can be symmetric or asymmetric, depending on the intended and desired convection and/or rinsing or flow of fluids or fluid mixtures within in the vessel.

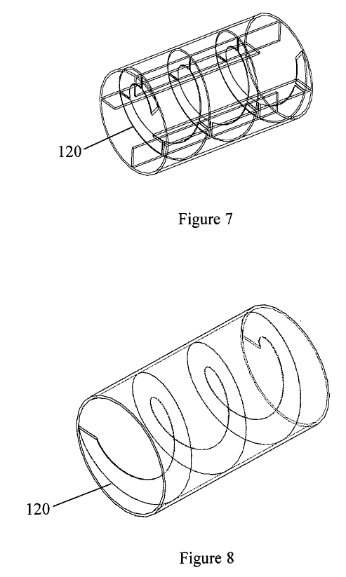

In further certain exemplary embodiments, a blade or a plurality of blades 120 can be helically wound along the inner vessel wall in any appropriate angle and direction, as shown in FIG. 7.

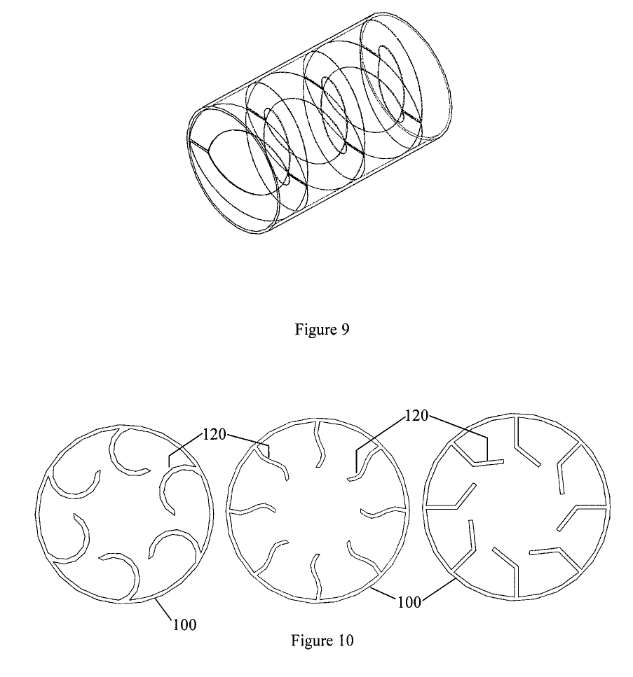

Furthermore, certain exemplary embodiments provide a plurality of helically winded blades, either in parallel or anti-parallel orientation or in any combination thereof, or in any non-parallel orientation, with or without intersecting a single blade or a plurality of blades. A single blade or a plurality of blades can fill the complete section of the inner vessel across the circumference or only specific sections, partially or completely or in any combination thereof as shown in FIGS. 8 and 9.

According to another exemplary embodiment of the present invention, any blade 120 can have a wave-like or undulating shape within its longitudinal direction or rectangular direction or in both directions, as shown in FIG. 10.

The wave can provide one peak as shown in FIG. 10, at a right drawing thereof, toward any direction, or a plurality of peaks with a serpentine-like form. For example, the linking struts can comprise at least one peak or one serpentine with two peaks. The orientation of the peaks or serpentines can be varied, e.g., a left-hand oriented peak or right-hand oriented serpentine with a right-hand oriented peak first and a right-hand oriented peak second or vice versa. In certain exemplary embodiments, the modified blades are all of the same design, in other exemplary embodiments, they can have alternating patterns or any different pattern or combination thereof. In further preferred exemplary embodiments, the lines towards the apex of a peak comprise also peaks or serpentines, either symmetrically or asymmetrically, and in further exemplary embodiments, at least one blade or a plurality of blades comprise any desired pattern of peaks and/or serpentines. According to one aspect of this exemplary embodiment, the design is not limited to one peak or one serpentine, e.g., it is also possible to embed a plurality of peaks and/or serpentines in any desired combination, whereby also the angles, curvatures and radiuses can be different individually within at least one blade or a plurality of blades. Peaks and serpentines can also be of angular-shape or varied in any desired geometric combination.

For example, the blade can be of angular cross-sectional geometry, the edges being rounded or not, but also specifically preferred are non-angular geometries.

The geometry can be identical or similar over the complete run or profile of a single blade, or different at any specific section or different at multiple sections. A plurality of blades can also comprise blades with different cross-sectional geometries.

The thickness of a blade can depend of the material and mechanical characteristics of the material, and preferably, the thickness can be selected appropriately to allow a fixed position or, if elastic movement is desired, to allow sufficient elastic movement.

Preferably, the blade(s) has/have a thickness in the range of about 0.0001 mm to 1,200 cm, more preferably in the range of about 0.01 mm to 10 cm, yet more preferably from about 0.1 mm to 5 cm and still yet more preferably from about 1 mm to 1 cm.

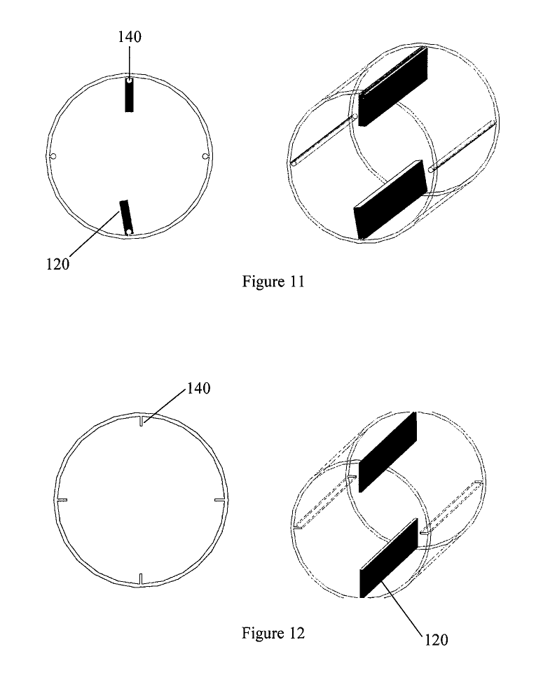

In other exemplary embodiments, a single blade 120 or a plurality of blades can have a connection that facilitates movement at least in one direction, preferably in any three-dimensional direction or in more than one three-dimensional direction. Preferably, the blade provides a joint. The joint 140 can be fixed to the blade holder 130 as defined below, and preferably provides a nodular end that is inserted into an appropriate cavity of the blade and allows movement, as shown in FIGS. 11 and 12 (see cross sections on the left drawings thereof). Any other suitable joint or connection 140 to the blade holder 120 conventionally known may be used to facilitate the aforesaid movement. Preferably, movement of the blade 120 can occur during the agitation of the fluid and/or vessel or blade holder by flowing and rinsing the liquid medium (passive moving). In further exemplary embodiments, the blade can be moved actively, for example by embedding a motor device and an axis that is connected to the blade. In these embodiments, the axis can be preferably sealed appropriately to avoid leakage.

According to one exemplary embodiment of the present invention, a single blade or a plurality of blades can have more than one connection that allows movement in one or more than one three-dimensional direction or any combination thereof and possibly preferred, with discontinuous blades.

In certain exemplary embodiments, it can be further preferred to have non-angular geometries of blades or plurality of blades. Suitable geometries are—in a cross-sectional view—semicircular geometries of any desired radius and dimension (see discussion herein), curvature, regularity or irregularity. According to the design of blades, a single blade or a plurality of blades can also have different radiuses, dimensions, curvatures or any combination thereof at different sections.

According to one exemplary embodiment, regular semi-circular geometries, or ladle-like geometries can be employed. In further exemplary embodiments, blades may be configured to hemispheric bowls that provide ladle-like surfaces. In certain exemplary embodiments, a blade or plurality of blades can be cross-sectional closed towards a circle, e.g., the geometry of a tube or tube-like form. This exemplary embodiment can be used with discontinuous blades. The tubes can have different dimensions, and possibly a capillary size, e.g., also at different sections.

Moreover, the blades (as described above) can comprise at least one tube-like hole, in particular a tube. Further, the blades can comprise more than one tube or tube-like or capillary form, hence a plurality of them. In addition, the plurality of tubes, tube-like or capillary forms may be of the same dimension and geometry, but in further exemplary embodiments, they can be different. Within a blade providing at least two tubes or tube-like or capillary forms, there can be interconnected, e.g., it may exit at least one connection between the at least two tubes or tube-like or capillary forms. The tubes, tube-like or capillary configuration of a blade may be designed to allow the uptake and/or through-flow of a fluid, i.e. the liquid medium or a gas or a gas mixture or any combination thereof, preferably during the agitation of the vessel or the inventive use of the vessel. Hence, the connection between the tubes, tube-like or capillary forms can facilitate the through-flow of the aforesaid fluid. According to an exemplary embodiment of the present invention, a plurality of blades can be provided with aforesaid tubes, tube-like or capillary design in any combination.

In further exemplary embodiments, a tube or tube-like or capillary blade can have a more complex design. For example, in further exemplary embodiments, the tube or tube-like or capillary form comprises at least another tube, tube-like or capillary form. Such constituted plurality of tubes or capillary can be arranged concentrically or eccentrically within each other or inside as a parallel oriented plurality or as a combination thereof, whether interconnected or not, of same or different geometry, size, diameter and so forth.

The exemplary embodiment of the described blades or pluralities of blades, independent of the geometry and orientation within the vessel, but particularly non-tubular or non-capillary designs of blades, can be hollow or comprise inside at least one tubular or any other cavity. For example, a blade can comprise a single capillary or a plurality of capillaries, interconnected or not, or a capillary system. An excavated tube or capillary or plurality of excavated tubes or capillaries can be oriented rectangular, parallel or in any three-dimensional orientation towards the vessel's longitudinal axis and/or towards each other's longitudinal axis.

In further exemplary embodiments, at least one blade can have at least one aperture at the basis that is oriented toward the vessel wall, optionally directly connected to the vessel wall or blade holder. The aperture can have a closing as described earlier above, preferably, a connection toward at least one different compartment inside the inventive vessel or outside of the vessel. Most preferably, the aperture is directly connected to at least one excavated capillary or tube within the aforesaid blade. Different apertures can be connected to different single or multiple compartments inside or outside of the inventive vessel or any combination thereof. The excavated blade, e.g., with at least one tube or capillary or capillary system, may be designed to provide or take up or release a fluid or fluid mixture, such as a gas or gas mixture, or a liquid or a liquid mixture or any combination thereof, that is either identical or different to the fluids or a part of the fluid comprised within the vessel, within at least one compartment of the vessel or at least one compartment outside of the inventive vessel or any combination thereof.

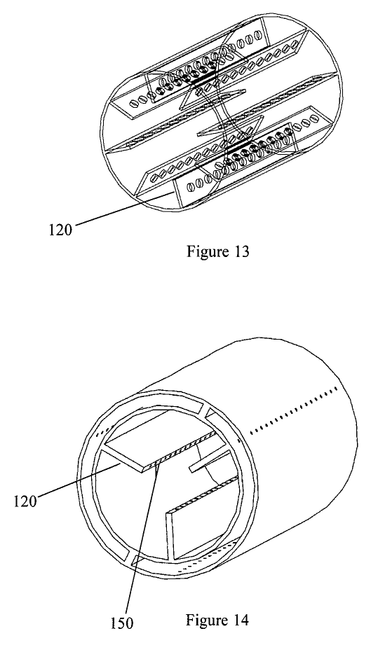

According to another exemplary embodiment of the present invention, the blade or plurality of blades can be perforated or comprise at least one tube-like hole 150, i.e. opening, or a plurality of tube-like holes, i.e. openings, as shown in FIGS. 13 and 14.

The perforation or tube-like hole, i.e. opening, connects the upper surface of a blade with the lower surface of the blade. The openings can have a round shape, ellipsoid shape, rectangular shape or any other regular or irregular geometry or any combination thereof.

In further exemplary embodiments, at least one opening, i.e. aperture, connects the surface of a blade with its cavity, excavated tube, or capillary or capillary system, or any combination thereof, as shown in FIG. 14. The openings, i.e. aperture, allow taking up, rinsing or releasing a fluid or a fluid mixture or any combination thereof.

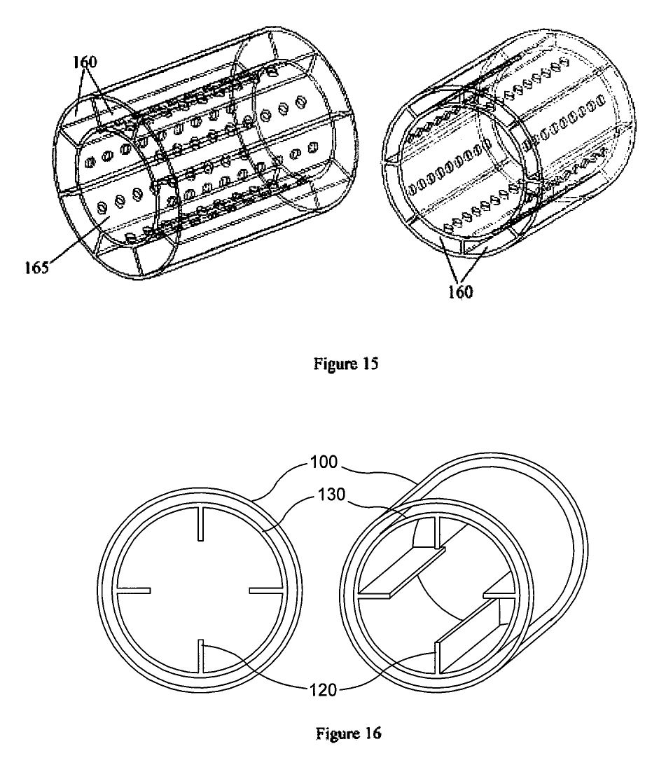

The holes may furthermore connect at least two different compartments or sectors 160, 165, within or outside or between inside and outside of the inventive vessel, as shown in FIG. 15. In certain exemplary embodiments, at least one hole or aperture can be closed with a closing as described earlier above, preferably with a valve.

The holes and/or openings may have an average diameter in the range of 0.5 to 100,000 μm, more preferably from 1 to 10,000 μm, still further preferable from 1,000 to 5,000 μm and yet further preferable from 10 to 100 μm.

In case of a capillary system said system has preferably, a volume in the range of 1 μl to 500 L, more preferably from 10 μl to 10 L, still further preferable from 10 μl to 1 L, yet further preferable from 1,000 μl to 1 L.

A plurality of blades can be connected together at any section or part of a single blade. Preferably, blades are connected directly to the inner vessel wall, but in some further exemplary embodiments, the blades are connected to a blade holder that is located with the vessel. It has to be noted that some information described herein concerning Figures showing vessels with blades may also apply to blade holders for insertion into vessels alone, since the structures can be similar, only the functions being different.

Exemplary Blade Holder

An exemplary embodiment of a blade holder can be located within the vessel, being not part of the vessel, being not a joint or connection between the vessel and the blade(s), optionally holding the blade(s) substantially in a predefined position from the inner surface of the vessel.

The blades connected to a blade holder can be oriented toward the outer surface or inner surface or both surface of the blade holder. The blade holder can be directly connected to the inner vessel wall, e.g., by clamping it into the vessel, or be without a direct connection to the inner vessel wall, and the connection can be fixed or not fixed. Preferably, the vessel comprises a cylindrical body so that the blade holder has also basically a cylindrical shape, for example a cylinder or ring that can be used within the inventive vessel. For example, the blade holder has substantially the same shape as the vessel but of smaller dimension. Or in other words, the blade holder has the same net shape of the vessel wherein the blade holder is used, for example, if the inventive vessel is of regular spherical shape then the blade holder also comprises the same spherical shape of a size that fits into the inventive vessel.

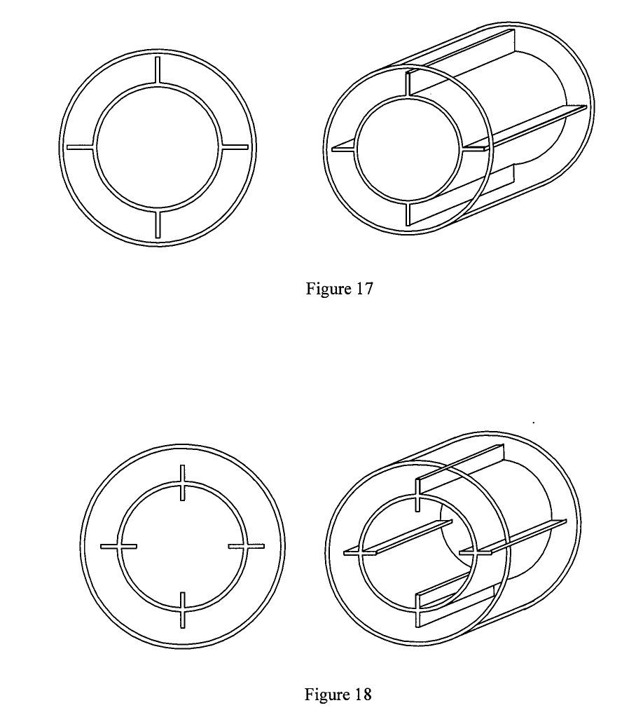

In certain exemplary embodiments, the blade holder can be a round slice or cylinder and has a diameter in the range of about 1.99 to 99.9 cm. Moreover, such exemplary blade holder may have a length in the range of about 1.99 to 319 cm. The exemplary blade holder can be made of a single part or out of multiple parts. For example, such blade holder 130 at least comprises one blade 120, more preferably, at least 2, 3 or 4 blades, as shown in FIGS. 16-18 and 20.

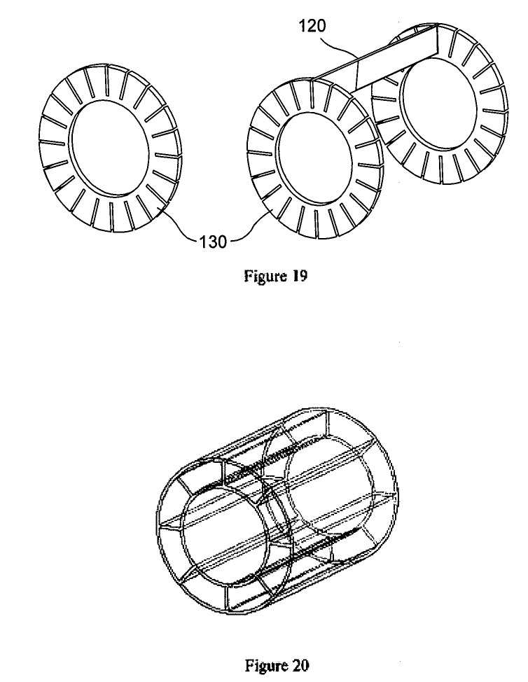

The exemplary blade holder can longitudinally fill the complete vessel or parts or sections of the vessel. Further, the blade holder can circumferentially fill substantially completely or partially the circumference or parts of the circumference of the vessel. A single blade or plurality of blades can be connected to more than one blade holder. The connection between a single or a plurality of blade holders 130 and plurality of blades 120 comprises a blade holder system as shown in FIG. 19. The inventive vessel can comprise more than one blade holder system, preferably, a plurality of different blade holders. The blade holder can comprise itself a plane cross-sectional or longitudinal geometry or any different regular or irregular geometry at any part, area or section in any three-dimensional direction. Preferably, the cross-sectional profile of the blade holder is undulating or providing wave-like structures with peaks and, more preferably, valleys or slots. In one exemplary embodiment of the present invention, the geometric structure of at least one blade holder comprises a plurality of regularly or irregularly patterned slots or cavities. Similarly to the blades, the at least one blade holder can comprise perforations or at least one opening, i.e. aperture, or a plurality of openings, i.e. aperture.

The perforation or opening, e.g., aperture, connects the inner surface of the blade holder with the outer surface of the blade holder. The openings, i.e. apertures, can have a round shape, ellipsoid shape, rectangular shape or any other regular or irregular geometry or any combination thereof. In further exemplary embodiments, at least one opening, i.e. aperture, connects the outer surface of a blade holder with a cavity, excavated tube, or capillary or capillary system of at least one blade or any combination thereof. The openings, i.e. apertures, allow taking up, rinsing or releasing a fluid or a fluid mixture or any combination thereof. The openings, i.e. apertures, furthermore connect at least two different compartments within or outside or between inside and outside of the inventive vessel. In certain exemplary embodiments, at least one opening, i.e. aperture, can be closed with a closing as described earlier above, preferably with a valve.

In other further exemplary embodiments, the blade holder comprises at least one hole, e.g., tube or tube-like or capillary form, hence a plurality of them in any combination thereof. For example, the plurality of holes, e.g., tubes, tube-like or capillary forms are of the same dimension and geometry, but in further exemplary embodiments, they are different. Within a blade holder providing at least two holes, i.e. tubes or tube-like or capillary forms there can be at least one connection between the at least two tubes or tube-like or capillary forms. The holes, e.g., tubes, tube-like or capillary configuration of a blade are designed to allow the uptake and/or through-flow of a fluid, i.e. the liquid medium or a gas or a gas mixture or any combination thereof, during the agitation of the vessel or the inventive use of the vessel. Thus, the connection between the holes, tubes, tube-like or capillary forms facilitates the through-flow of the fluid. According to the exemplary embodiment of the present invention, there can be also a plurality of blade holders with aforesaid holes, i.e. tubes, tube-like or capillary design in any combination.

In further exemplary embodiments, a tube or tube-like or capillary blade holder can have a more complex design. For example, in further exemplary embodiments, the tube or tube-like or capillary form comprises at least another tube, tube-like or capillary form. The so constituted plurality of tubes or capillary can be arranged concentrically or eccentrically within each other or inside as a parallel oriented plurality or any combination thereof, whether interconnected or not, of same or different geometry, size, diameter, and so forth.

The exemplary blade holder or plurality of blade holders, independent of the geometry and orientation within the vessel, but particularly non-tubular or non-capillary designs of blade holders, can be hollow or comprise inside at least one tubular or any other cavity. For example, the blade holder can comprise a single capillary or a plurality of capillaries, interconnected or not, or a capillary system. An excavated tube or capillary or plurality of excavated tubes or capillaries can be oriented rectangular, parallel or in any three-dimensional orientation towards the vessel's longitudinal axis and/or towards each other's longitudinal axis.

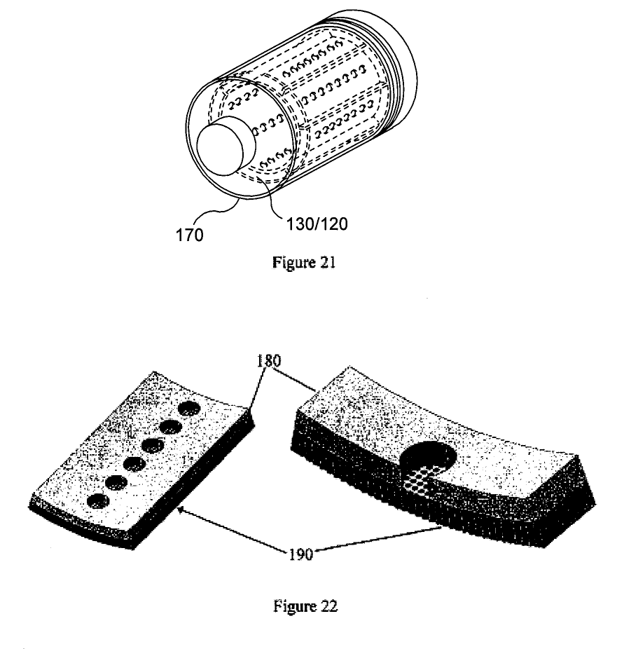

In further exemplary embodiments, at least one blade holder has at least one aperture that is oriented toward the vessel wall, or at least one connected blade or both, optionally directly connected, see e.g., FIG. 21 illustrating a roller bottle 170 including a convection arrangement 130/120. The aperture can have a closing as described earlier above, preferably, a connection toward at least one different compartment inside the inventive vessel or outside of the vessel or to a blade or excavated part of a blade or any combination thereof.

For example, the aperture can be directly connected to at least one excavated capillary or tube within at least one blade. Different apertures can be connected to different single or multiple compartments inside or outside of the inventive vessel or inside or outside of a single or multiple compartments of at least one blade or any combination thereof. The excavated blade holder, e.g., with at least one tube or capillary or capillary system, is designed to provide or take up or release a fluid or fluid mixture, like a gas or gas mixture, or a liquid or a liquid mixture or any combination thereof, that is either identical or different to the fluids or a part of the fluid comprised within the vessel, within at least one compartment of the vessel or at least one compartment outside of the vessel or any combination thereof.

At least a part of at least one of the convection arrangement, the blade holder or a blade can be made of a porous material, with ultramicro-porous, micro-porous or meso-porous or macro-porous or combined pores or porosities. These can be completely or partially porous at any section or part or at different sections or parts. The average pore sizes can preferably be in a range of about 2 Angström up to 1,000 μm, further preferable from about 1 nm to 800 μm. Furthermore, these components can be completely or partially porous selectively on the inner or outer or both surfaces, or completely throughout the body of the part. The porous components of the convection arrangement can comprise a gradient of different porous layers or sections in any desired geometric or three-dimensional direction. In further exemplary embodiments, the porous structure can be partially or completely a mesh-like porous structure or a lattice, and/or comprises a mesh-like trabecular, regular or irregular or random or pseudo-random, structure or any combination thereof or the aforesaid porous structures, essentially having the same pore sizes as mentioned above. In certain exemplary embodiments, a blade, plurality of blades or blade holder can comprise two or more different layers with different designs, for example a first layer 180 with large pores connected to a second layer 190 with a plurality of capillaries or tubular cavities, as shown e.g., in FIG. 22.

In certain exemplary embodiments, it may be possible to fix a blade holder or a blade holder system by just clamping it inside of the inventive vessel. Clamping can be realized by designing the size of the blade holder or blade holder system that it is self-fixing, sometimes preferably with introducing at least one discontinuous space holder, for example a protrusion like a pin or a flange, or at least one continuous space holder like a flanged ring, either at the outer surface or circumference of the blade holder or blade holding system or at the inner surface of the inventive vessel or both. Any other method conventionally known can be applied. Other suitable methods can include, but not limited to, bonding or welding of the parts, or screwing.

In further exemplary embodiments, the blade holder or blade holding system can be fixed laterally at least at one point or part or section at the cross-sectional plane. Generally, according to an exemplary embodiment of the present invention, fixation can be realized indicated herein, and further, the fixation may facilitate a centric or eccentric rotation around the longitudinal axis of the blade holder or blade holding system or around any other or a plurality of three-dimensional axis.

In further exemplary embodiments, it can be preferable to fix the blade holder or blade holding system at least one perforation or aperture to at least one corresponding perforation or opening of the inventive vessel, for example, by welding or bonding, or further preferable by a conventional connection, like an inlet, valve, hollow screws, tubes or tubing or any combination thereof. The fixation at least at one single point or part can be embedded anywhere at the circumference of the blade holder or blade holder system or at the cross-sectional plane at one or both lateral ends of the blade holder or blade holder system and/or inventive vessel. In other further exemplary embodiments, at least one blade holder or at least one blade holding system or a plurality of both aforesaid are not fixed within the inventive vessel. Most preferred, the fixation is designed to connect at least one aperture and/or opening of the inventive vessel with at least one aperture or opening of the blade holder or blade holding system. In a further exemplary embodiment, this fixation allows the rotation of at least the blade holder or blade holding system. In certain exemplary embodiments of the present invention, the rotation can be actively enabled by directly or indirectly coupled drive or similar procedure pr arrangement known in the art.

In an additional exemplary embodiment of the present invention, the blade holder or respective blade holding system can have a vessel-like design, preferably, a cylindrical body, but not limited to, whereby the cylindrical body has at least one aperture on one lateral end that allows filling in a liquid medium and/or cell suspension, e.g., using a pipette, and a second lateral end that is closed or optionally comprises also at least one aperture. The aperture is preferably centered to the longitudinal axis of the blade holder or blade holding system, but in some further exemplary embodiments, the aperture or respective apertures can be eccentric. The shape of the aperture can be round, but in certain exemplary embodiments, it is possible to have rectangular or any other regular or irregular shape of the aperture. The aperture or respective apertures can be closed and opened reversibly, e.g., by a closing like a screw cap requiring an appropriate thread, for example by comprising a threaded neck. In further exemplary embodiments, the apertures can have a neck to take the screw cap, but any other known closing to reversibly close or open the aperture can be used. In certain exemplary embodiments, the vessel is narrowed toward the aperture or the respective neck comprising the aperture, in further exemplary embodiments both lateral ends are narrowed. In some certain exemplary embodiments, the aperture may not be embedded into the lateral ends, but preferably, at the central body. In further exemplary embodiments, more than one aperture is comprised at the vessel body, optionally any combination of at least one lateral aperture and at least one aperture at the body of the vessel.

In further exemplary embodiments, the closing of at least one aperture comprises a membrane for gas exchange as known in the art with appropriate sealing against leakage of the liquid medium. In further exemplary embodiments, the closing of at least one aperture can be opened to allow for gas exchange without using a membrane.

In further exemplary embodiment, at least one of the closings comprises a valve, either for unidirectional in-flow or out-flow of fluids like liquids or gases or both, or bi-directional flow of fluids. Optionally, more closings provide valves in any desired combination. The valves can be pressure-sensitive, or a modulating valve, can be activated by mechanical means, electromechanical means or magnetically or by any appropriate procedure or arrangement known in the art. In further exemplary embodiments, at least one used closing comprises an aperture, either centric or eccentric, or optionally more than one aperture. These apertures can comprise closings that can be reversibly opened or closed, for example screw caps or valves or the like or any combination thereof. The closings used, for any aperture, can also comprise rotating joints or swivel couplings, optionally with valves, for example to connect a tube or tubing to the aforesaid apertures.

In one further exemplary embodiment of the present invention, the exemplary blade holder or blade holding system can be used with an inventive vessel comprising directly connected blades or pluralities of blades. For example, the directly connected blades are located in a specific circumferential section of the vessel and the blade holder or blade holding system is located side by side to the section with directly connected blades. In certain exemplary embodiments, more than one section of the vessel comprises directly connected blades and one or a plurality of blade holders or blade holding systems is introduced additionally, either in an alternating pattern or in any different regular or irregular pattern. In further exemplary embodiments, at least one blade holder can be nested into a vessel comprising at least one directly connected blade or a plurality of directly connected blades. In further exemplary embodiments, any combination of the aforesaid design can be embedded. In further exemplary embodiments, the nested blade holder or blade holding system may comprise at least one or more additionally nested blade holder or blade holding system into the foregoing.

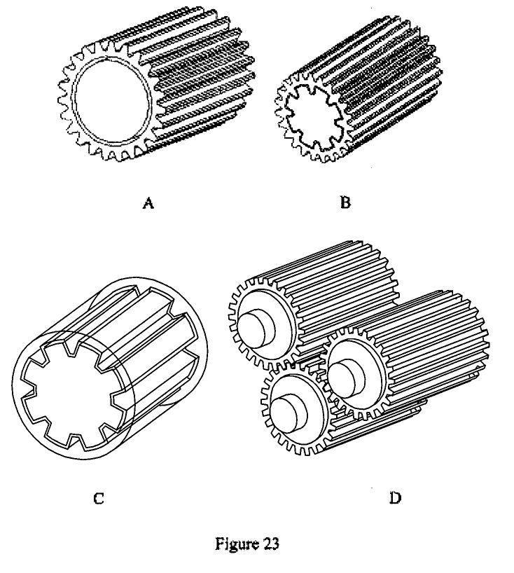

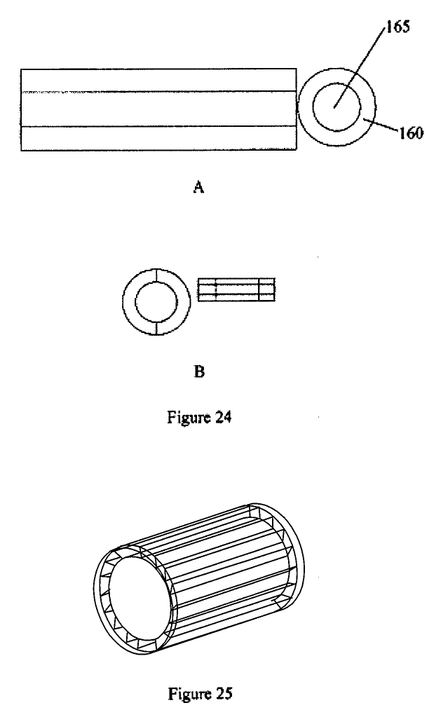

In certain exemplary embodiments, the vessel can provide a cross-sectional blade pattern like a cogwheel as shown in FIG. 23D, either with a screw-like or helically run or not, and the inserted blade holder or blade holding system can comprise at the outer circumferential surface blades also with a corresponding cross-sectional pattern like a cogwheel, either with a screw-like or helically run or not, as shown in FIGS. 23A-D. In more certain exemplary embodiments, the blade holder may comprise at the inner circumferential surface a cross-sectional blade pattern like a cogwheel, either with a screw-like or helically run or not, as shown in FIG. 23C. In certain exemplary embodiments, where the vessel comprises a cross-sectional blade pattern like a cogwheel, certain exemplary embodiments of the blade holder or blade holding system comprise on both the outer and inner circumferential surface blades also with a corresponding cross-sectional pattern like a cogwheel, as shown in FIG. 23B. On both circumferential surfaces, cogwheel patterned blade holder or blade holding system can be used to nest further cogwheel patterned blade holders or blade holding systems inside, etc.

The exemplary nested blade holders or blade holding systems can be nested into the vessel or in each other centrically or eccentrically or in any combination. Cogwheel-like blade design and different blade designs or blade holder or blade holding system designs can be implemented in any combination within the same vessel. The cogwheel-like design may be preferred in a cultivation system, where the agitation of the culture is partially or mainly carried out by rotating the vessel or at least one blade holder or blade holding system or any combination thereof. The number and distances of cogwheel-like blades or the pattern design allows tailoring the transmission of the rotation and respective rotation speed to the desired conditions.

Exemplary Rotatable Vessels

The vessel, e.g., a cylindrical body, can have a plane wall, in certain exemplary embodiments, it may be preferred to comprise a wall with a regular or irregular pattern of undulating wave-like peaks or cavities. Preferably, the cross-sectional profile or the longitudinal profile or any combination thereof is undulating or providing wave-like structures with peaks and, more preferably, valleys or slots. In another exemplary embodiment of the present invention, the geometric structure of at least one part or section of the vessel body comprises a plurality of regularly or irregularly patterned slots or cavities.

In further exemplary embodiment of the present invention, the vessel comprises throughout the wall or only at the outer layer of the wall at least at one circumferential part a cogwheel like pattern of cogs. The circumferential design of a cog-pattern can facilitate the rotation of the vessel around its longitudinal axis by a cogwheel-like roller with an appropriate apparatus. In further exemplary embodiments, the circumferential section of cog-like wall design is covering the complete vessel surface, as shown in FIG. 23D. In other exemplary embodiments, the vessel comprises a plurality of circumferential cogwheel like pattern of cogs with identical or different patterns. Generally, the cylindrical vessel may comprise at least one arrangement of cavities and/or elevations in substantially steady distances and said arrangement is located around the outer surface of the cylindrical vessel in a direction parallel to the longitudinal axis of the cylindrical vessel. The exemplary arrangement of cavities and/or elevations typically extends in longitudinal direction over the whole length, or at least a part of the vessel, and may be one of a wave-like pattern, a cogwheel-like pattern, a screw-like or a helical run, as desired to allow rotation of the vessel, preferably of a plurality of vessels contacting each other, as shown in FIG. 23D.

Exemplary Compartmented Vessels

In further exemplary embodiment of the present invention, e.g., at least two compartments or sectors 160/165, can be provided within the vessel, as shown in FIG. 24. The compartments can be oriented parallel to the cross-sectional plane of the vessel or longitudinal plane of the vessel or to any other three-dimensional plane. The compartments or sectors can be identically in volume or size, symmetrically or asymmetrically. The compartments can also be comprised by a vessel design with at least two or more nested geometrically identically shaped but appropriately sized parts that are closed at the ends, such as concentric cylinders. Most preferred are cylindrical bodies or any combination thereof or the foregoing.

Furthermore, it is possible to include more than two compartments or sectors, as shown in FIGS. 20 and 25. The two compartments or at least two compartments of a plurality of compartments can be separated from each other (see FIG. 20), by the wall facilitating fluid communication between the sectors. Each single wall can have at least one aperture that allows filling in a liquid medium and/or cell suspension, e.g., using a pipette. The aperture is preferably centered to the longitudinal axis of the vessel, but in some further exemplary embodiments, the aperture or respective apertures can be eccentric or is located at any optional position within the separating wall. The shape of the aperture is most preferably round, and in certain exemplary embodiments, it is possible to have rectangular or any other regular or irregular shape of the aperture. The aperture or respective apertures can be closed and opened reversibly, e.g., by a closing like a screw cap requiring an appropriate thread, for example by comprising a threaded neck. In further exemplary embodiments, the apertures have a neck to take the screw cap, but any other known closing to reversibly close or open the aperture can be used. In further exemplary embodiments more than one aperture is comprised at the separating wall, cf. FIG. 25.

In further exemplary embodiments, the closing of at least one aperture comprises a membrane for gas exchange as known in the art with appropriate sealing against leakage of the liquid medium. In other further exemplary embodiments, the closing of at least one aperture can be opened to allow for gas exchange without using a membrane.

In further exemplary embodiments, at least one of the closings comprises a valve, either for unidirectional in-flow or out-flow of fluids like liquids or gases or both, or bi-directional flow of fluids. Optionally, more closings provide valves in any desired combination. The valves can be pressure-sensitive, or a modulating valve, can be activated by mechanical means, electromechanical means or magnetically or by any appropriate arrangement or technique known in the art. In further exemplary embodiments, at least one used closing comprises an aperture, either centric or eccentric, or optionally more than one aperture. These apertures also comprise closings that can be reversibly opened or closed, for example screw caps or valves or the like or any combination thereof. The closings used, for any aperture, can also comprise rotating joints or swivel couplings, optionally with valves, for example to connect a tube or tubing to the aforesaid apertures.

In further exemplary embodiments, at least two apertures of different compartments are connected to each other using tubing or a tube. Preferably, in further exemplary embodiments, at least one aperture of a separating wall is connected to an aperture or opening of a blade holder or blade holding system or a single blade or a plurality of blades.

In further exemplary embodiments, at least one separating wall of two compartments or sectors is porous, with ultramicro-porous, micro-porous or meso-porous or macro-porous or combined pores or porosities. A separating wall can completely or partially be porous at any section or part or at different sections or parts. Furthermore, a separating wall or plurality of separating walls can be completely or partially porous selectively on the inner or outer or both surfaces, or completely throughout the body of the part. The porous separating wall can comprise a gradient of different porous layers or sections in any desired geometric or three-dimensional direction. In some further exemplary embodiments, the porous structure is partially or completely a mesh-like porous structure or a lattice, or comprises a mesh-like trabecular, regular or irregular or random or pseudo-random, structure or any combination thereof or the aforesaid porous structures. In further exemplary embodiments, the separating wall of two compartments comprises a membrane, either completely or partially.

In further exemplary embodiments, a blade or a blade holder or a blade holding system or any combination thereof may be designed to constitute at least a separating wall and/or a second compartment or a plurality of separating walls and/or compartments.

In certain exemplary embodiments, independent of the geometry and size of the vessel, it is preferred to provide a vessel that is hollow or comprises inside of the wall at least one tubular or any other cavity. For example, a vessel wall can comprise a single tube and/or capillary or a plurality of tubes and/or capillaries, interconnected or not, or a tubular and/or capillary system. An excavated tube or capillary or plurality of excavated tubes or capillaries can be oriented rectangular, parallel or in any three-dimensional orientation towards the vessel's longitudinal axis and/or towards each other's longitudinal axis.

In further exemplary embodiments, the vessel wall has at least one capillary or tube with an aperture that is oriented towards the outer or inner surface of the vessel wall or both, optionally directly connected but not necessarily. The aperture can have a closing as described earlier above, e.g., a connection toward at least one different compartment inside the inventive vessel or outside of the vessel or to a compartment or a plurality of compartments, or a blade or excavated part of a blade or any combination thereof. For example, the aperture can be directly connected to at least one excavated capillary or tube within at least one blade or compartment. Different apertures can be connected to different single or multiple compartments inside or outside of the inventive vessel or inside or outside of a single or multiple compartments of at least one blade or blade holder or any other combination thereof. The excavated vessel wall, e.g., with at least one tube or capillary or capillary system, is designed to provide or take up or release a fluid or fluid mixture, like a gas or gas mixture, or a liquid or a liquid mixture or any combination thereof, that is either identical or different to the fluids or a part of the fluid comprised within the vessel, within at least one compartment of the vessel or at least one compartment outside of the inventive vessel or any combination thereof.

In further exemplary embodiments, the vessel wall can be porous, with ultramicro-porous, micro-porous or meso-porous or macro-porous or combined pores or porosities having pore sizes as described below. A vessel wall can be completely or partially porous at any section or part or at different sections or parts. Furthermore, a vessel wall can be completely or partially porous selectively on the inner or outer or both surfaces, or completely throughout the body of the part. The porous vessel wall can comprise a gradient of different porous layers or sections in any desired geometric or three-dimensional direction. In certain exemplary embodiments, the porous structure can be partially or completely a mesh-like porous structure or a lattice, or comprises a mesh-like trabecular, regular or irregular or random or pseudo-random, structure or any combination thereof or the aforesaid porous structures. In further exemplary embodiments, the vessel wall may comprise either partially or completely a membrane.

The exemplary cavity or interconnected may have a volume in the range of at least about 0.01%, preferably about 0.01 to 99%, more preferably in the range about 1 to 50% and yet more preferably in the range about 25 to 80% of the overall vessel volume.

The surface area of the interior of the vessel can be increased by the blade(s) and optionally by the blade holder by a factor of about 0.8·10¹⁰ to 20·10¹⁰, and preferably of about 1.2·10¹⁰ to 6·10¹⁰.

For example, in case of a porous or porous-like material as described herein, the vessel wall, the blade(s) and/or the blade holder can comprise at least partially a macro-porous, meso-porous, micro-porous or ultra-microporous material or any combination thereof, whereby the pore sizes may be preferably in a range of about 2 Angstrom up to about 1,000 μm, and further preferable from about 1 nm to 800 μm.

For example, in case of a mesh-like or lattice-like material as defined in the instant invention, the vessel wall, the blade(s) and the blade holder is comprised at least partially by a mesh-like or lattice-like material, whereby the average size between the mesh size is preferably in a range of about 2 Angstrom up to 1000 μm, and further preferable from about 1 nm to 800 μm.

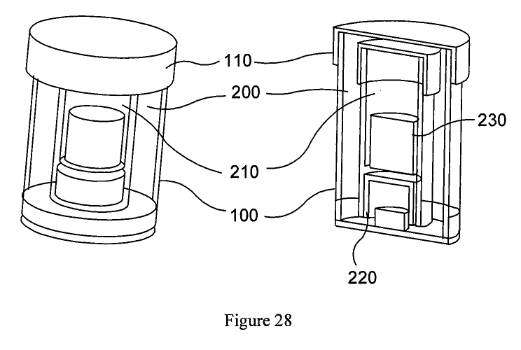

Another exemplary embodiment according to the present invention is shown in FIG. 28. In this exemplary embodiment, a vessel 100 is provided with a cap 110, either with or without gas exchange membrane, that consists of three compartments. In one exemplary configuration, the first compartment, e.g., the outer compartment is free of any filler, whereas the second compartment 210 comprises packed discrete filler materials 230. Additionally, a third compartment 220 (e.g., the bottom compartment) can contain the convection arrangement, e.g., a magnetic stirrer in the center of the said compartment. The bottom compartment can be connected by at least one whole to the inner compartment 210 and by at least one, and preferably by two, three or more holes to the outer compartment 200. According to another exemplary embodiment, the vessel provides a reverse configuration, e.g., to insert the filler 230 into the first, e.g., outer compartment 200.

Exemplary Modular Vessels

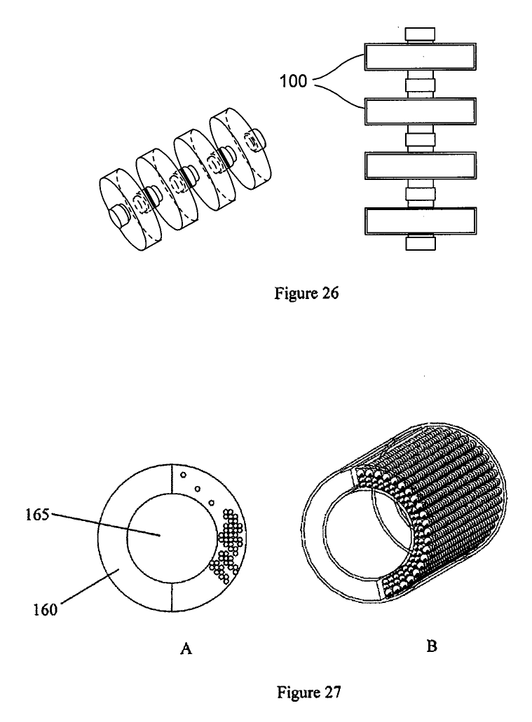

In further exemplary embodiments, the vessel comprises a plurality of connected compartments or sectors. In these exemplary embodiments, each vessel comprises a design as described above and can be used as a cultivation vessel stand-alone. Optionally, a second vessel can be connected or a plurality of vessels can be connected, as shown in FIG. 26. The exemplary connection can comprise at least one closing as described above with a rotating joint or swivel coupling, optionally with valves, connected either by a tube or tubing or directly connected to each other. Exemplary vessels can have a discoid geometry with at least one aperture and connecting closing to each other that is centric to the longitudinal axis of the discs. More specifically, the connection facilitates the rotation of both discoid vessels synchronous or asynchronous, in the same direction or opposite directions, with the same speed or different speeds. Further exemplary embodiments comprise at least one circumferential section with cogwheel-like cogs at the outer surface of the vessel wall at least of one discoid vessel, but specifically preferred at all vessels. The exemplary pattern of the cogwheel design can be identical or different. The exemplary agitation of the vessel is then a rotation around the longitudinal axis, whereby at least a single roller with a corresponding design transmits the rotation to the vessel. It is possible to drive the connected discoid vessels independently with different speeds and directions or even selectively not to move a single or specific number of discoid vessels.

Exemplary Convection and Rinsing System

The exemplary function of the exemplary embodiment of the convection and rinsing system can be to provide sufficient exchange and supply of medium, medium compounds, fluids and fluid mixtures. For example, in the conventional systems, nutritional supply may be affected by increasing cell mass and not appropriately addressed by sufficient convection. With the exemplary embodiment of the cultivation system, it is feasible to provide at any point and compartment of the system sufficient nutritional compounds, beneficial agents and or fluids or fluid mixtures as well as a high surface area for physiological exchange of compounds, e.g., supply of nutritional compounds and removal of intermediates. The rinsing system can be designed to selectively supply fluids or fluid mixtures, e.g., medium that can be rinsed by droplet formation in order to increase further the overall surface of the liquid fluids for enhanced gas exchange. By increasing also the overall cross-section of fluid providing compartments, the pressure can be reduced below critical values to protect the cells, tissues or tissue-like cell cultures, organs or organ-like cell cultures, multicellular organisms from shear stress or any pressure induced damages. The exemplary embodiment of the convection system can have the function to optimally distribute the flow of fluids within a single or plurality of compartments through the complete cultivation system. The exemplary patterns of convection may be unilateral confection, multi-circular convection, and/or spiral convection.

Depending on the blade configuration, any desired convection pattern can be realized.

Exemplary Preferred Materials

The exemplary embodiment of the cultivating system can be manufactured in one seamless part or with seams out of multiple parts. The exemplary cultivation system may be manufactured using known manufacturing techniques. A further option may be to weld individual sections together. Any other suitable manufacturing process may also be applied and used.

Any part that is used according to the exemplary cultivation system, including filler, can be made from a suitable material conventionally used, as desired, e.g., partially or completely made by conventional means of polymers, glass, ceramics, composites, metals, metal alloys or any mixture thereof, e.g., metals and metal alloys selected from main group metals of the periodic system, transition metals such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals. For the vessel, transparent polymeric materials may be sometimes preferred, whereas for the convection arrangement, blades, blade holders, and fillers materials having acceptable properties as a substrate for cell growth may be preferred, particularly biocompatible, optionally even biodegradable materials. The material can be selected from any suitable metal or metal oxide or shape memory alloys any mixture thereof to provide the structural body of the implant. For example, the material is selected from the group of zero-valent metals, metal oxides, metal carbides, metal nitrides, metal oxynitrides, metal carbonitrides, metal oxycarbides, metal oxynitrides, metal oxycarbonitrides and the like, and any mixtures thereof. The metals or metal oxides or alloys used in a further exemplary embodiment of the present invention may be magnetic. Examples are—without excluding others—iron, cobalt, nickel, manganese and mixtures thereof, for example iron, platinum mixtures or alloys, or for example, magnetic metal oxides like iron oxide and ferrite.

It may be preferred to use semi-conducting materials or alloys, for example semi-conductors from Groups II to VI, Groups III to V, and Group IV. Suitable Group II to VI semi-conductors are, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, or mixtures thereof. Examples for suitable Group III to V semi-conductors are GaAs, GaN, GaP, GaSb, InGaAs, InP, InN, InSb, InAs, AlAs, AlP, AlSb, AlS and mixtures thereof. Examples for Group IV semi-conductors are germanium, lead and silicon. The semi-conductors may also comprise mixtures of semi-conductors from more than one group and all the groups described above are included.

In further exemplary embodiments, the material can be made of biodegradable metals which can include, e.g., metals, metal compounds such as metal oxides, carbides, nitrides and mixed forms thereof, or metal alloys, e.g., particles or alloyed particles including alkaline or alkaline earth metals, Fe, Zn or Al, such as Mg, Fe or Zn, and optionally alloyed with or combined with other particles selected from Mn, Co, Ni, Cr, Cu, Cd, Pb, Sn, Th, Zr, Ag, Au, Pd, Pt, Si, Ca, Li, Al, Zn and/or Fe. Further suitable may be, e.g., alkaline earth metal oxides or hydroxides such as magnesium oxide, magnesium hydroxide, calcium oxide, and calcium hydroxide or mixtures thereof. In exemplary embodiments, the biodegradable metal-based particles may be selected from biodegradable or biocorrosive metals or alloys based on at least one of magnesium or zinc, or an alloy comprising at least one of Mg, Ca, Fe, Zn, Al, W, Ln, Si, or Y. Furthermore, the implant may be substantially completely or at least partially degradable in-vivo. Examples for suitable biodegradable alloys can comprise, e.g., magnesium alloys comprising more than 90% of Mg, about 4-5% of Y, and about 1.5-4% of other rare earth metals such as neodymium and optionally minor amounts of Zr; or biocorrosive alloys comprising as a major component tungsten, rhenium, osmium or molybdenum, for example alloyed with cerium, an actinide, iron, tantalum, platinum, gold, gadolinium, yttrium or scandium.

In further exemplary embodiments, the material may be selected from organic materials. Preferred materials are biocompatible polymers, oligomers, or pre-polymerized forms as well as polymer composites. The polymers used may be thermosets, thermoplastics, synthetic rubbers, extrudable polymers, injection molding polymers, moldable polymers, spinnable, weavable and knittable polymers, oligomers or pre-polymerizes forms and the like or mixtures thereof. In certain exemplary embodiments, it is useful to select the material from biodegradable organic materials, for example—without excluding others—collagen, albumin, gelatine, hyaluronic acid, starch, cellulose (methylcellulose, hydroxypropylcellulose, hydroxypropylmethylcellulose, carboxymethylcellulose-phtalate); furthermore casein, dextrane, polysaccharide, fibrinogen, poly(D,L lactide), poly(D,L-lactide-Co-glycolide), poly(glycolide), poly/hydroxybutylate), poly(alkylcarbonate), poly(orthoester), polyester, poly(hydroxyvaleric acid), polydioxanone, poly(ethylene, terephtalate), poly(maleic acid), poly(tartaric acid), polyanhydride, polyphosphohazene, poly(amino acids), and all of the copolymers and any mixtures thereof.

In certain exemplary embodiment, the material can be based on inorganic composites or organic composites or hybrid inorganic/organic composites. The material can also comprise organic or inorganic micro- or nano-particles or any mixture thereof. Preferably, the particles used in the present invention are selected from the group of zero-valent metals, metal oxides, metal carbides, metal nitrides, metal oxynitrides, metal carbonitrides, metal oxycarbides, metal oxynitrides, metal oxycarbonitrides and the like, and any mixtures thereof. The particles used in a further exemplary embodiment of the present invention may be magnetic. Examples are—without excluding others—iron, cobalt, nickel, manganese and mixtures thereof, for example iron, platinum mixtures or alloys, or for example, magnetic metal oxides like iron oxide and ferrite. It may be preferred to use semi-conducting particles, for example semi-conductors from Groups II to VI, Groups III to V, and Group IV. Suitable Group II to VI semi-conductors are, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, or mixtures thereof. Examples for suitable Group III to V semi-conductors are GaAs, GaN, GaP, GaSb, InGaAs, InP, InN, InSb, InAs, AlAs, AlP, AlSb, AlS and mixtures thereof. Examples for Group IV semi-conductors are germanium, lead and silicon.

In yet another further exemplary embodiment, the materials may be selected from polymers, oligomers or pre-polymeric particles. Examples of suitable polymers for use as particles in the present invention are homopolymers, copolymers, prepolymeric forms and/or oligomers of poly(meth)acrylate, unsaturated polyester, saturated polyester, polyolefines like polyethylene, polypropylene, polybutylene, alkyd resins, epoxy-polymers or resins, phenoxy polymers or resins, phenol polymers or resins, polyamide, polyimide, polyetherimide, polyamideimide, polyesterimide, polyesteramideimide, polyurethane, polycarbonate, polystyrene, polyphenole, polyvinylester, polysilicone, polyacetale, cellulosic acetate, polyvinylchloride, polyvinylacetate, polyvinylalcohol, polysulfone, polyphenylsulfone, polyethersulfone, polyketone, polyetherketone, polybenzimidazole, polybenzoxazole, polybenzthiazole, polyfluorocarbons, polyphenylenether, polyarylate, cyanatoester-polymere, and mixtures of any of the foregoing.

Furthermore, polymer materials may be selected from oligomers or elastomers like polybutadiene, polyisobutylene, polyisoprene, poly(styrene-butadiene-styrene), polyurethanes, polychloroprene, or silicone, and mixtures, copolymers and combinations of any of the foregoing.

In a certain exemplary embodiment, the materials can be selected from electrically conducting polymers, preferably from saturated or unsaturated polyparaphenylene-vinylene, polyparaphenylene, polyaniline, polythiophene, poly(ethylenedioxythiophene), polydialkylfluorene, polyazine, polyfurane, polypyrrole, polyselenophene, poly-p-phenylene sulfide, polyacetylene, monomers oligomers or polymers thereof or any combinations and mixtures thereof with other monomers, oligomers or polymers or copolymers made of the above-mentioned monomers. Particularly preferred are monomers, oligomers or polymers including one or several organic, for example, alkyl- or aryl-radicals and the like or inorganic radicals, like for example, silicone or germanium and the like, or any mixtures thereof. Preferred are conductive or semi-conductive polymers having an electrical resistance between 1012 and 1012 Ohm·cm. It may be preferred to select those polymers which comprise complexed metal salts.

In another exemplary embodiment, the materials are selected from biodegradable materials like for example—without excluding others—collagen, albumin, gelatine, hyaluronic acid, starch, cellulose (methylcellulose, hydroxypropylcellulose, hydroxypropylmethylcellulose, carboxymethylcellulose-phtalate); furthermore casein, dextrane, polysaccharide, fibrinogen, poly(D,L lactide), poly(D,L-lactide-Co-glycolide), poly(glycolide), poly/hydroxybutylate), poly(alkylcarbonate), poly(orthoester), polyester, poly(hydroxyvaleric acid), polydioxanone, poly(ethylene, terephtalate), poly(maleic acid), poly(tartaric acid), polyanhydride, polyphosphohazene, poly(amino acids), and all of the copolymers and any mixtures thereof.

Exemplary Cultivation Process

The exemplary vessels and systems described herein can be used in the exemplary cultivation process in which at least one type of cells, tissue, tissue-like cell cultures, organs, organ-like cell cultures, or multicellular organisms are cultivated, e.g., grown and harvested, in the presence of at least one fluid or solid medium necessary for growing and/or cultivating the aforesaid culture. This can be done in a conventional manner, e.g., by using a suitable fluid medium in the vessel. For example, the medium can be a liquid such as water, and may comprise at least one of proteins, polypeptides, peptides, oligopeptides, carbohydrates, glycoproteins, glycopeptides, glycolipids, lipids, fatty acids, lipoproteins, glycolipids, glucose, fructose, peptone, ammonium salts, magnesium, potassium salts, natrium salts. Also, the medium can be gaseous and may comprise at least one of CO₂, CO, oxygen, N₂, NO, NO₂, N₂O, hydrogen, or SO₂ or any mixture thereof.

The liquid medium may comprise between about 0.1 to 100%, further preferable from about 20 to 70% and most preferred about 30 to 60% of the vessel volume.

In a further exemplary embodiment, the liquid medium and/or gaseous medium can be provided in at least one capillary system or excavation or any combination thereof, and can be continuously or discontinuously rinsing and/or flowing through at least one capillary system or cavity. In one embodiment of the cultivation process, the culture vessel comprises at least one filler that releases a biologically active agent, either temporarily or continuously. In another exemplary embodiment of the cultivation process, the culture vessel comprises at least one filler that absorbs one compound comprised or released by the cultivated cells, tissue, tissue-like cell cultures, organs, organ-like cell cultures, or multicellular organisms. In one embodiment of the cultivation process, the culture vessel comprises at least one filler that releases at least one signal generating that is attaching to or incorporated into the cultivated cells, tissue, tissue-like cell cultures, organs, organ-like cell cultures, or multicellular organisms. In a further exemplary embodiment of the cultivation process, the culture vessel comprises at least one filler that releases at least one virus, virus particle, vector, DNA or any other agent that is useful for transfection of the cultivated cells, tissue, tissue-like cell cultures, organs, organ-like cell cultures, or multicellular organisms. In still another exemplary embodiment of the cultivation process, the culture vessel comprises at least one filler that is used as a carrier for temporarily or permanent attachment of cells, tissue, tissue-like cell cultures, organs, organ-like cell cultures, or multicellular organisms.

In a further exemplary embodiment of the cultivation process, the culture vessel comprises at least one filler that is used to buffer the pH of the culture medium between pH 3 to pH 12, further preferable from pH 5 to 9 and most preferred from pH 6 to 8.

In a still further exemplary embodiment of the cultivation process, the culture vessel is rotated continuously or discontinuously with a rotating speed of 0.01 rpm to 10 rpm, further preferable from 0.1 rpm to 6 rpm and most preferred from 0.5 rpm to 6 rpm. In an additional exemplary embodiment of the cultivation process, the culture vessel is shaken continuously or discontinuously with a speed of about 0.01 rpm to 10 rpm, further preferable from about 0.1 rpm to 6 rpm and most preferable from about 0.5 rpm to 6 rpm. In another exemplary embodiment of the cultivation process, the culture vessel is teetered continuously or discontinuously in an angle of about 0.1° to 350°, further preferable from about 10° to 45°, with a speed of about 0.01 rpm to 10 rpm, further preferable from about 0.1 rpm to 6 rpm and most preferable from about 0.5 rpm to 6 rpm.

In a further exemplary embodiment of the cultivation process, the liquid and/or gaseous medium is rinsing or flowing continuously or discontinuously throw at least one filler comprising at least one flow-channel. In still another exemplary embodiment of the cultivation process, the liquid medium is continuously or discontinuously pumped into and/or out of the vessel, one compartment of the vessel or capillary system or excavation or any combination thereof with a flow rate between 0.0001 ml/min and 10,000 ml/min, further preferable between 0.001 ml and 100 ml/min and most preferred between 1 ml and 10 ml.

In still another exemplary embodiment of the cultivation process, the gaseous medium is continuously or discontinuously pumped into and/or out of the vessel, one compartment of the vessel or capillary system or excavation or any combination thereof with a pressure between about −1,000 and 10,000 mbar, further preferable between about −0.001 and 1,000 mbar and most preferable between about 1 and 10 mbar.

In an exemplary embodiment of the cultivation process, the gaseous medium is continuously or discontinuously flowing into and/or out the concentration of CO₂ within the gas phase is kept constantly by using the at least one absorptive filler in a range of about 1% to 90%, further preferable between about 1% to 20% and most preferable between about 4% and 6%.

In yet another exemplary embodiment of the cultivation process, the cells and/or compounds released by the cells, tissue, tissue-like cell cultures, organs, organ-like cell cultures, or multicellular organisms are discontinuously or continuously removed out of the vessel, a compartment, a capillary or excavation by at least partial outflow of liquid medium. In still further exemplary embodiment of the cultivation process, the cells and/or compounds released by the cells, tissue, tissue-like cell cultures, organs, organ-like cell cultures, or multicellular organisms are discontinuously or continuously removed out of the vessel, a compartment, a capillary or excavation by at least partially removing a filler. In still another exemplary embodiment of the cultivation process, the cells, tissue, tissue-like cell cultures, organs, organ-like cell cultures, or multicellular organisms are discontinuously or continuously removed out of the vessel, a compartment, a capillary or excavation by at least partially removing a filler.

It should be noted that the term ‘comprising’ does not exclude other elements or steps and the ‘a’ or ‘an’ does not exclude a plurality. In addition elements described in association with the different embodiments may be combined.

It should be noted that the reference signs in the claims shall not be construed as limiting the scope of the claims.

Having thus described in detail several exemplary embodiments of the present invention, it is to be understood that the present invention described above is not to be limited to particular details set forth in the above description, as many apparent variations thereof are possible without departing from the spirit or scope of the present invention. The exemplary embodiments of the present invention are disclosed herein or are obvious from and encompassed by the detailed description. The detailed description, given by way of example, but not intended to limit the present invention solely to the specific embodiments described, may best be understood in conjunction with the accompanying Figures.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the present invention.

What is claimed is:

1. A reversibly closable vessel for cultivation of at least one of cells or tissues, comprising:
(i) a wall provided in an interior of the vessel and having therein at least one reversibly closable aperture; the wall being integral to the vessel and dividing the interior into at least two compartments, wherein the wall comprising at least one of a hole, an opening or a partially porous structure facilitating a flow of fluids between the compartments; and
(ii) at least one filler provided in at least one of the compartments.

2. The vessel of claim 1, further comprising a convection arrangement provided inside the vessel, the convection arrangement including at least one of a magnetic stirring bar, an agitator, a stirrer, and at least one blade optionally fixed to a blade holder, the convention arrangement being capable to at least of generate or modify a convection in a fluid within the vessel when at least one of the fluid, the vessel or the blade is agitated.

3. The vessel of claim 2, wherein the at least one blade comprises:
i. at least one cavity, and
ii. at least one aperture or partially porous structure on the surface of said blade connecting the cavity with said surface.

4. The vessel of claim 3, wherein at least one of the at least one cavity comprises the filler.

5. The vessel of claim 1, wherein the filler is densely packed in at least one of the compartments.

6. The vessel of claim 1, wherein the vessel has a rotational symmetric shape.

7. The vessel of claim 1, wherein the vessel has a cylindrical shape.

8. The vessel of claim 1, wherein the vessel has a shape of a roller bottle.

9. The vessel of claim 2, wherein the at least one blade is connected to an inner portion of the wall or is integral with the vessel.

10. The vessel of claim 2, wherein the at least one blade is connected to a blade holder, the holder being:
i. located within the vessel,
ii. unintegral with the vessel,
iii. not a joint or connection between the vessel and the blade, and
iv. optionally holding the at least one blade substantially in a predefined position from an inner surface of the vessel.

11. The vessel of claim 10, wherein the wall in the interior surface of the vessel is the blade holder.

12. The vessel of claim 10, wherein the blade holder has an inner surface and an outer surface.

13. The vessel of claim 12, wherein the at least one blade is fixed to the outer surface or the inner surfaces of the blade holder.

14. The vessel of claim 10, wherein the blade holder has a rotational symmetric shape.

15. The vessel of claim 10, wherein the blade holder has a cylindrical shape.

16. The vessel of claim 10, wherein the blade holder has a shape that is substantially identical or similar to a shape of the vessel with smaller dimensions.

17. The vessel of claims 14, wherein the blade holder is a round slice or a cylindrical shape.

18. The vessel of claim 1, further comprising at least one blade is connected to an inner portion of the wall or is integral with the vessel, and a blade holder being:
i. located within the vessel,
ii. unintegral with the vessel,
iii. not a joint or connection between the vessel and the blade, and
iv. optionally holding the at least one blade substantially in a predefined position from an inner surface of the vessel.

19. The vessel of claim 2, wherein the at least one blade is fixed substantially perpendicular to at least one of a surface of the vessel or a surface of the blade holder.

20. The vessel of claim 12, wherein the at least one blade comprises a plurality of blades, wherein at least two of the blades located on the outer surface of the blade holder extend to the inner surface of the vessel to produce the compartments in between the inner and other surfaces.

21. The vessel of claim 20, wherein the blades extending from the outer surface of the blade holder to the inner surface of the vessel are fixed on the surfaces.

22. The vessel of claim 2, wherein at least one of the vessel wall, the at least one blade or the blade holder has at least one of a hole or a opening facilitating the flow of fluids there through.

23. The vessel of claim 1, wherein the hole or opening is interconnected.

24. The vessel of claim 1, wherein the hole or opening is tube-like interconnected.

25. The vessel of claim 24, wherein the hole or the opening forms a capillary system.

26. The vessel of claim 22, wherein at least one of the hole, the opening of the vessel wall, the at least one blade or the blade holder is interconnected with at least another one of the hole or the opening of another one of the group consisting of the vessel wall, the at least one blade or the blade holder.

27. The vessel of claim 1, wherein at least one of the wall, the at least one blade or the blade holder has at least one cavity.

28. The vessel of claim 1, wherein the wall, the at least one blade or the blade holder comprises at least two cavities being interconnected with one another.

29. The vessel of claim 28, wherein at least one of the cavities has a volume at least about 0.01% of an overall vessel volume.

30. The vessel of claim 28, wherein at least one of the cavities has a volume in the range of about 0.01 to 99% of an overall vessel volume.

31. The vessel of claim 28, wherein at least one of the cavities has a volume in the range of about 1 to 50% of an overall vessel volume.

32. The vessel of claim 28, wherein at least one of the cavities has a volume in the range of about 25 to 80% of an overall vessel volume.

33. The vessel of claim 27, wherein at least one of the wall, the at least one blade or the blade holder has at least one closable aperture on a surface connecting the at least one cavity with the surface.

34. The vessel of claim 1, wherein at least one of the wall, the at least one blade or the blade holder is at least partially made from a porous material, a macro-porous material, a meso-porous material, a micro-porous material or a ultra-microporous material or any combination thereof.

35. The vessel of claim 1, wherein at least one of the wall the at least one blade or the blade holder is at least partially made of a mesh-like material or a lattice-like material.

36. The vessel of claim 33, wherein the wall is provided for a capillary system which at least partially comprises a membrane.

37. The vessel of claim 27, wherein the wall of the at least one cavity at least partially comprises a membrane.

38. The vessel of claim 1, wherein the wall and the at least one aperture in the wall comprises at least one of a closing or a valve.

39. The vessel of claim 1, wherein wall or the at least one aperture in the wall is of a dimension facilitating an insertion of a blade holder into an interior of the vessel.

40. The vessel of claim 39, wherein the at least one aperture is located on a base of the vessel.

41. The vessel of claim 1, wherein an overall available surface area of an interior of the vessel is increased by the filler by a factor of about 1.1 to 20·10¹⁰.

42. The vessel of claim 1, wherein an overall available surface area of an interior of the vessel is increased by the filler by a factor of about 1.2 to 6·10¹⁰.

43. The vessel of claim 1, wherein an overall available surface area of an interior of the vessel is increased by the filler by a factor of about 2.0 to 5·10⁵.

44. The vessel of claim 1, wherein the at least one filler has at least one of a high surface area, an absorption capacity or a buffering capacity.

45. The vessel of claim 1, wherein the at least one filler comprises particles with a size ranging from about 1 nm up to 1 cm.

46. The vessel of claim 1, wherein the at least one filler comprises particles with a size ranging from about 2 nm up to 2000 μm.

47. The vessel of claim 1, wherein the at least one filler comprises particles with a size ranging from about 10 nm up to 1000 μm.

48. The vessel of claim 1, wherein the at least one filler comprises particles with a size ranging from about 200 nm up to 800 μm.

49. The vessel of claim 1, wherein the at least one filler comprises a structured mould having at least one flow-channel.

50. The vessel of claim 49, wherein a diameter of the at least one flow-channel is in a range of about 100 nm up to 20 cm.

51. The vessel of claim 49, wherein a diameter of the at least one flow-channel is in a range of about 100 μm to 1 cm.

52. The vessel of claim 49, wherein a diameter of the at least one flow-channel is in a range of about 500 μm to 8 mm.

53. The vessel of claim 49, wherein a diameter of the at least one flow-channel is in a range of about 1,000 to 5,000 μm.

54. The vessel of claim 1, wherein the at least one filler is made at least partially from a macro-porous material, a meso-porous material, a micro-porous material or an ultra-microporous material, or any combination thereof.

55. The vessel of claim 1, wherein the at least one filler is made at least partially from a mesh-like material or a lattice-like material.

56. The vessel of claim 1, wherein the at least one filler is selected from the group consisting of fillers capable for adherent cell growth, fillers for increase of the surface area for equilibrium, fillers suitable for exchange of fluids or fluid mixtures, fillers suitable for exchanging anions or cations or both, absorbents for fluids that provide a nutritional compound or a plurality of nutritional compounds, fillers for selectively adsorbing or desorbing physiologically or biologically active agents.

57. The vessel of claim 1, wherein the at least one filler is located within at least one cavity of at least one of the wall, the at least one blade or the blade holder.

58. The vessel of claim 1, wherein at least one filler releases an agent.

59. The vessel of claim 58, wherein the agent is selected from any of the groups comprising a biologically agent, a pharmacologically, therapeutically or adsorptive agent or a signal generating agent.

60. The vessel of claim 1, wherein the at least one compartment comprises at least one ion exchanger as a filler.

61. The vessel of claim 1, wherein at least one compartment comprises at least one absorber as a filler, the absorber being selected from the group consisting of
(i) an absorber capable of absorbing at least one compound of a culture,
(ii) an absorber capable of absorbing a fluid, a mixture of the fluid or a component of the fluid, or
(iii) or mixtures thereof.

62. The vessel of claim 61, wherein at least one of the absorbers is selected from the group consisting of gas absorbing materials.

63. The vessel of claim 61, wherein at least one of the absorbers is selected from the group consisting of at least one of an absorption of CO₂, oxygen, N₂, NO, NO₂, N₂O or SO or any combination thereof.

64. The vessel of claim 61, wherein the absorber and the ion exchanger are located in different compartments.

65. The vessel of claim 1, wherein the blade is actively moved by embedding a motor device in the vessel, the motor device comprises an axis that is connected to the at least one blade.

66. The vessel of claim 6, wherein the cylindrical vessel comprises at least one arrangement of at least one of cavities or elevations in substantially steady distances, the at least one arrangement being located around an outer surface of the cylindrical vessel in a direction parallel to a longitudinal axis of the cylindrical vessel.

67. The vessel of claim 66, wherein the at least one arrangement extends in a longitudinal direction over an entire length of the vessel.

68. The vessel of claim 66, wherein the at least one arrangement is of one of a wave-like pattern, a cogwheel-like pattern, a screw-like run or a helical run.

69. A system comprising:
at least two reversibly closable vessels for cultivation of at least one of cells or tissues, at least one of the vessels comprising:
i. a wall provided in an interior of the vessel and having therein at least one reversibly closable aperture; the wall being integral to the vessel and dividing the interior into at least two compartments, wherein the wall comprising at least one of a hole, an opening or a partially porous structure facilitating a flow of fluids between the compartments; and
ii. at least one filler provided in at least one of the compartments.
wherein the vessels are interconnected via the at least one aperture in the wall.

70. The system according to claim 69, wherein the vessels are independently rotatable.

71. A procedure for providing or implementing at least one reversibly closable vessel for cultivation of at least one of cells or tissues, comprising:
providing or utilizing the vessel which comprises:
i. a wall provided in an interior of the vessel and having therein at least one reversibly closable aperture; the wall being integral to the vessel and dividing the interior into at least two compartments, wherein the wall comprising at least one of a hole, an opening or a partially porous structure facilitating a flow of fluids between the compartments, and
ii. at least one filler provided in at least one of the compartments; and
cultivating cells, tissues, tissue-like cell cultures, organs, organ-like cell cultures or multicellular organisms using the vessel.

72. A cultivation process using at least one reversibly closable vessel for cultivation of at least one of cells or tissues, comprising:
providing the vessel which comprises:
i. a wall provided in an interior of the vessel and having therein at least one reversibly closable aperture; the wall being integral to the vessel and dividing the interior into at least two compartments, wherein the wall comprising at least one of a hole, an opening or a partially porous structure facilitating a flow of fluids between the compartments, and
ii. at least one filler provided in at least one of the compartments; and
cultivating cells, tissues, tissue-like cell cultures, organs, organ-like cell cultures or multicellular organisms using the vessel in a presence of at least one fluid or a solid medium for at least one of growing or cultivating the culture.
